(19) United States
(12) Patent Application Publication
(10) Pub. No.: US 2024/0315981 A1
(43) Pub. Date: Sep. 26, 2024

(54) SUB-MICRON PARTICLE

(71) Applicant: IMPERIAL COLLEGE INNOVATIONS LIMITED, London (GB)

(72) Inventors: Rongjun Chen, London (GB); Xuhan Liu, London (GB); Robin Shattock, London (GB); Anna Blakney, London (GB); Yifan Liu, London (GB)

(21) Appl. No.: 18/570,611

(22) PCT Filed: Jun. 14, 2022
(86) PCT No.: PCT/GB2022/051493

§ 371 Date: Dec. 14, 2023

(30) Foreign Application Priority Data
Jun. 14, 2021 (GB) 2108444.7

(51) Int. Cl. A61K 9/51; A61K 39/00; A61K 39/145; A61K 39/215; A61P 37/04

(52) U.S. Cl. CPC A61K 9/5123; A61K 9/5146; A61K 9/5192; A61K 39/145; A61K 39/215; A61P 37/04; A61K 2039/53

(57) ABSTRACT

The invention relates to a sub-micron particle comprising a payload molecule and a lipid structure being surrounded by an outer layer comprising an amphiphilic copolymer. The invention extends to method of producing the sub-micron particle. The invention also encompasses pharmaceutical compositions and vaccines comprising the sub-micron particle and medical uses thereof.

The present invention relates to sub-micron particles, and in particular to sub-micron particles per se comprising a payload molecule, such as a nucleic acid, or small molecule drug. The invention extends to methods of producing the sub-micron particles, pharmaceutical compositions and vaccines comprising the sub-micron particles, and to medical uses thereof.

Vaccines are one of the most cost-effective methods to prevent infectious diseases. The World Health Organization reported that vaccinations could prevent two to three million deaths per year. Traditional vaccines involve attenuated virus or purified signature proteins of the virus. More recently, however, RNA vaccines are promising candidates due to their rapid development and low-cost manufacture. They are among global frontrunners in the race to clinical trials against COVID-19, including the already approved messenger RNA (mRNA) vaccines of Pfizer/BioNTech and Moderna, as well as the Imperial College's self-amplifying RNA (saRNA) vaccines in clinical trials. RNA vaccines have also been used preclinically for a variety of other vaccine indications, including infectious diseases such as influenza¹, rabies virus², HIV-1 3, Zika virus 4, and Ebola virus⁵, as well as for cancer vaccines⁶⁻⁹. mRNA-based vaccines typically encode the antigen of interest and contain 5′ and 3′ untranslated regions (UTRs), whereas saRNA-based vaccines encode not only the antigen but also the viral replication machinery that enables intracellular RNA amplification and abundant protein expression¹⁰. saRNA enable a large amount of antigen production from an extremely small dose (10- to 100-fold lower than mRNA) owing to intracellular replication of the antigen-encoding RNA¹¹. The dose-sparing quality of saRNA vaccines may facilitate scale-up and manufacturing large numbers of vaccine doses.

Although mRNA and saRNA hold great promise as a new class of vaccines and therapeutics, their clinical translation and commercialization are still limited due to two big challenges. One challenge is the difficulty on intracellular delivery problems, since the RNA have: (1) insufficient endocytosis on account of its large molecular weight and negative charge which induces repulsion to cell membrane, (2) limited intracellular protein expression due to catalytic hydrolysis/enzymolysis caused by endosomal entrapment and (3) inadequate antigen loading and maturation of antigen-presenting cells (APCs).

The other major problem is that RNA is very fragile and may readily degrade in exposure environments, thus requiring RNA vaccines to be stored and transported in a very challenging cold chain. Any break of “cold chain” would decrease RNA vaccine's efficacy significantly. For example, Pfizer/BioNTech's mRNA vaccine, the world's first approved COVID-19 vaccine, is plagued by the major hurdle requiring storage at −70° C. At refrigerated temperatures of 2-8° C., the mRNA vaccine can only be stable for only 5 days (Pfizer.com, 20/11/2020). Similarly, Moderna's mRNA already approved for COVID-19 needs to be held in storage at −20° C. This makes it extremely challenging for RNA vaccines to reach the required speed and scale of deployment for vaccinations.

Lyophilization may increase the RNA stability by avoiding aqueous conditions. However, the formed ice crystal during lyophilization exhibits physical stress on the nucleic acid (sequence) and other components of the formulation, which may lead to a damage of the nucleic acid (e.g., breakage of strands, loss of supercoiling, etc.) and irreversible aggregation or precipitation of the RNA-loaded formulation. All of these will lead to an irreversibly decreased efficacy of RNA formulations and limit the rapid development of RNA vaccines and therapeutics.

Therefore, development of stable nucleic acid vaccines and therapeutics (be they saRNA, mRNA or DNA) at 2-8° C. and non-cold chain (in particular the latter) is of importance for supply, distribution and deployment, but highly challenging. Given all these challenges, it is of unprecedented urgency to develop a novel platform with low cost and safety profile for efficient nucleic acid delivery and enhanced stability, not only at refrigerated temperatures, but even at ambient temperatures.

In the past several decades, advances in bioengineering and nanotechnology have produced a number of delivery technologies. Liposomes as the widely studied RNA delivery system need to be positively charged to electrostatically trap RNA, and a certain membrane charge density threshold has been identified as a requirement to ensure efficient endosomal escape¹²⁻¹⁴. However, payloads in liposomes are easily leaked, and the lipid membrane can be disrupted when interacting with the negatively charged cell membranes. Besides, the surface charge of liposomes will affect their aggregation behaviour as well as the adsorption of serum proteins once injected in vivo. These will cause the fast clearance of RNA and reduced in vivo transfection efficiency. The use of amphiphilic diblock copolymers to generate polymer vesicles known as polymersomes is another strategy to create structures for encapsulation. Polymersomes are also studied for oligonucleotides delivery by researchers as they have higher mechanical strength and toughness than liposomes^{15,16}. However, the synthesis of cationic polymers requires quite tricky procedures due to the complicated biological requirements. Besides, it is more difficult for efficient delivery of mRNA or saRNA due to the much bigger molecular weight and instability of RNA than oligonucleotides.

Although some reported formulations showed efficient mRNA or saRNA delivery efficacy^{17,18}, the thermal stability is still challenging as they were used fresh or kept at −80° C. for use later.

The present invention arises from the inventors' work in attempting to overcome the problems associated with the prior art.

In accordance with a first aspect of the invention, there is provided a sub-micron particle comprising a payload molecule and a lipid structure, being surrounded by an outer layer comprising an amphiphilic copolymer.

Advantageously, the inventors were surprised to observe that the sub-micron particle may be used for efficient nucleic acid delivery (including saRNA, mRNA or DNA) and non-cold chain storage. This delivery system simultaneously addresses many if not all of the intended design requirements, including good biocompatibility, easy to manufacture, compact size, controlled surface charge, high RNA loading efficiency, endosomolytic capability, low cost and superior stability. Furthermore, the sub-micron particle may be produced using FDA-approved amphiphilic polymers (e.g., PEG-PCL) and cationic or ionizable lipids. Both materials are of low cost and readily available.

The amphiphilic copolymer forms a capsule, which serves as colloidal stable shell. The lipid structure can be self-assembled into aggregates, which are wrapped in the core of the capsule. The nucleic acid, such as RNA (e.g., mRNA or saRNA), can be encapsulated efficiently in this nanocontainer by electrostatic interaction with the cationic or ionizable lipid. The sub-micron particle provides dual protections for RNA: (1) the efficient condensation of RNA, and (2) the outer vesicular membrane, which is of higher mechanical stability and lower permeability than lipid bilayer. Compared to the current reported lipid nanoparticles, the colloidal stability of the sub-micron particles can be significantly enhanced as the mechanical strength of hydrophilic shell composed of polymer is much higher than that composed of lipid bilayer, allowing for better protection of the RNA during storage and applications.

Furthermore, advantageously, the sub-micron particles can be prepared by a one-pot method based on several minutes' mixing, stirring and solvent evaporation. The physicochemical and biological properties (e.g., particle size, surface charge, transfection efficiency and stability) of the sub-micron particles can be easily regulated by simply changing the mixing ratios.

The term “sub-micron” can be understood to mean that the particle of the invention has a largest maximum dimension of less than 1 μm. More preferably, the maximum dimension of the particle is less than 900 nm, less than 800 nm, less than 700 nm or less than 600 nm, and most preferably less than 500 nm, less than 400 nm, less than 300 nm or less than 200 nm. The sub-micron particle may have a largest maximum dimension of between 10 and 900 nm, between 20 and 800 nm, between 30 and 700 nm or between 40 and 600 nm, more preferably between 50 and 500 nm or between 60 and 400 nm, and most preferably between 80 and 300 nm or between 100 and 200 nm. The largest maximum dimension of the sub-micron particle may correspond to the Z-average size as determined using Zetasizer V instrument.

The payload molecule may be encapsulated by the lipid structure.

The payload molecule may be a biomolecule and/or an active pharmaceutical ingredient (API). The API may be a hydrophobic or hydrophilic API. The API may be a macromolecule or a small molecule. It may be appreciated that a small molecule could be considered to be a molecule with a molecular weight of less than 900 daltons. In some embodiments, a small molecule may have a molecular weight of less than 800 daltons, less than 700 daltons, less than 600 daltons, less than 500 daltons or less than 400 daltons. Similarly, a macromolecule may be considered to be a molecule with a molecular weight of at least 900 daltons.

In a preferred embodiment, the payload molecule is a biomolecule. For instance, the biomolecule may be or comprise an amino acid, a peptide, an affimer, a protein, a glycoprotein, a lipopolysaccharide, an antibody or a fragment thereof, or a nucleic acid.

The nucleic acid may be DNA, RNA or a DNA/RNA hybrid sequence. Preferably, the nucleic acid is DNA or RNA.

Most preferably, the nucleic acid is RNA. The RNA may be single stranded or double stranded. The RNA may be selected from the group consisting of: messenger RNA (mRNA); self-amplifying RNA (saRNA); antisense RNA (asRNA); RNA aptamers; interference RNA; micro RNA (miRNA); short interfering RNA (siRNA); short hairpin RNA (shRNA); and small RNA.

Preferably, the RNA is self-amplifying RNA (saRNA) or messenger RNA (mRNA). The skilled person would appreciate that self-amplifying RNAs may contain the basic elements of mRNA (a cap, 5′ UTR, 3′UTR, and poly(A) tail of variable length), but may be 20 considerably longer (for example 9-12 kb).

The nucleic acid sequence, preferably RNA, may be at least 10 bases in length, at least 20 bases in length, at least 50 bases in length, at least 100 bases in length, at least 200 bases in length, at least 300 bases in length, at least 400 bases in length, at least 500 bases in length, at least 600 bases in length at least 700 bases in length, at least 800 bases in length or at least 900 bases in length. In one preferred embodiment, the RNA is saRNA or mRNA.

The nucleic acid sequence, preferably RNA, and most preferably saRNA or mRNA, may be at least 1000 bases in length, at least 2000 bases in length, at least 3000 bases in length, at least 4000 bases in length, at least 5000 bases in length, at least 6000 bases in length, at least 7000 bases in length, at least 8000 bases in length, at least 9000 bases in length at least 10000 bases in length, at least 11000 bases in length or at least 12000 bases in length.

In one embodiment, the nucleic acid sequence is at least 6000 bases in length. In one embodiment, the RNA is at least 6000 bases in length. In a preferred embodiment, the saRNA is at least 6000 bases in length.

In an alternative embodiment, the nucleic acid sequence is at least 900 bases in length. In one embodiment, the RNA is at least 900 bases in length. In a preferred embodiment, the mRNA is at least 900 bases in length.

The nucleic acid sequence, preferably RNA, and most preferably saRNA, may be between 5000 and 20000 bases in length, between 5000 and 15000 bases in length, between 5000 and 14000 bases in length, between 500o and 13000 bases in length, between 500o and 12000 bases in length, between 5000 and 11000 bases in length, between 5000 and 10000 bases in length, between 6000 and 20000 bases in length, between 6000 and 15000 bases in length, between 6000 and 14000 bases in length, between 6000 and 13000 bases in length, between 6000 and 12000 bases in length between, between 6000 and 11000 bases in length, between 6000 and 10000 bases in length, between 7000 and 20000 bases in length, between 7000 and 15000 bases in length, between 7000 and 14000 bases in length, between 7000 and 13000 bases in length, between 7000 and 12000 bases in length, between 7000 and 11000 bases in length, between 7000 and 10000 bases in length, between 8000 and 20000 bases in length, between 8000 and 15000 bases in length, between 8000 and 14000 bases in length, between 8000 and 13000 bases in length, between 8000 and 12000 bases in length, between 8000 and 11000 bases in length, between 8000 and 10000 bases in length, between 9000 and 20000 bases in length, between 9000 and 15000 bases in length, between 9000 and 14000 bases in length, between 9000 and 13000 bases in length, between 9000 and 12000 bases in length, between 9000 and 11000 bases in length or between 9000 and 10000 bases in length.

Alternatively, the nucleic acid sequence, preferably RNA, and most preferably mRNA, may be between 50 and 10000 bases in length, between 100 and 9000 bases in length, between 200 and 8000 bases in length, between 300 and 7000 bases in length, between 400 and 6000 bases in length, between 500 and 6000 bases in length, between 600 and 5000 bases in length, between 700 and 4000 bases in length, between 800 and 3000 bases in length or between 900 and 2000 bases in length.

In one embodiment, the nucleic acid sequence is between 6000 and 15000 bases in length. The nucleic acid sequence may be between 8000 and 12000 bases in length. The RNA may be between 6000 and 15000 bases in length. The RNA may be between 8000 and 12000 bases in length. Preferably, the saRNA is between 6000 and 15000 bases in length. Preferably the saRNA is between 8000 and 12000 bases in length.

In an alternative embodiment, the nucleic acid sequence is between 400 and 14000, between 500 and 10000, between 600 and 7500, between 700 and 5000, between 800 and 4000 or between 900 and 2000 bases in length. The RNA may between 400 and 14000, between 500 and 10000, between 600 and 7500, between 700 and 5000, between 800 and 4000 or between 900 and 2000 bases in length. Preferably, the mRNA is between 400 and 14000, between 500 and 10000, between 600 and 7500, between 700 and 5000, between 800 and 4000 or between 900 and 2000 bases in length.

The skilled person would appreciate that when the nucleic acid is double stranded, for example double stranded RNA, “bases in length” will refer to the length of base pairs.

The nucleic acid may encode at least a portion of a virus. The virus may be the SARS-CoV-2 virus or an influenza virus. The nucleic acid may encode a SARS-CoV-2 spike protein, more preferably a pre-fusion stabilized SARS-CoV-2 spike protein.

Alternatively, the nucleic acid may encode as the H₁ hemagglutinin of the influenza virus. In some embodiments, the nucleic acid is RNA. In some embodiments, the nucleic acid is saRNA or mRNA.

The sub-micron particle preferably comprises a plurality of lipid structures. For instance, the sub-micron particle may comprise at least 1, at least 2, at least 3, at least 4, at least 5, at least 6, at least 7, at least 8, at least 9 or at least 10 lipid structures. The plurality of lipid structures may be surrounded by the outer layer comprising the amphiphilic copolymer.

The or each lipid structure may be a lipid nanoparticle or a liposome. Preferably, the or each lipid structure is a lipid nanoparticle.

The or each lipid structure may comprise a cationic or ionizable lipid. In some embodiments, the or each lipid structure may comprise a plurality of lipids. At least one of the plurality of lipids may comprise a cationic or ionizable lipid. The cationic or ionizable lipid may be a multivalent cationic lipid. The cationic or ionizable lipid may be a pH-sensitive lipid. The cationic or ionizable lipid may comprise a positively charged or ionizable nitrogen atom. The cationic or ionizable lipid may display a positive charge in an acidic solution. A solution may be understood to be acidic if it has a pH of less than 7 at 20° C., more preferably less than 6.5 at 20° C. A solution may be understood to be acidic if it has a pH of between 3.5 and 7 at 20° C. or between 4 and 7 at 20° C., more preferably between than 4.5 and 6.5 at 20° C. The cationic or ionizable lipid may be dioleoyl-3-trimethylammonium propane (DOTAP), 1,2-di-O-octadecenyl-3-trimethylammonium propane (DOTMA), an ethylphosphatidylcholine (ethyl PC), didodecyldimethylammonium bromide (DDAB), 3B-[N-(N′,N′-dimethylaminoethane)-carbamoyl]cholesterol (DC-Cholesterol), N₄-Cholesteryl-Spermine (GL₆₇), 1,2-dioleyloxy-3-dimethylaminopropane (DODMA), DLin-MC₃-DMA, 1,2-dioleoyl-3-dimethylammonium-propane (DODAP), or heptadecan-9-yl 8-((2-hydroxyethyl) (6-oxo-6-(undecyloxy) hexyl) amino) octanoate (SM-102).

In some embodiments, the lipid structure may comprise a cationic or ionizable lipid, such as DOTAP. The lipid structure may comprise at least 1 wt %, at least 10 wt %, at least 20 wt %, at least 30 wt %, at least 40 wt %, at least 50 wt %, at least 60 wt %, at least 70 wt %, at least 80 wt %, at least 90 wt % or at least 99 wt % cationic or ionizable lipid. In some embodiments, the lipid structure may consist of a cationic or ionizable lipid. Alternatively, the lipid structure may comprise between 1 and 99 wt % cationic or ionizable lipid, between 10 and 90 wt % cationic or ionizable lipid, between 20 and 85 wt % cationic or ionizable lipid, between 30 and 80 wt % cationic or ionizable lipid, between 40 and 75 wt % cationic or ionizable lipid, between 50 and 70 wt % cationic or ionizable lipid or between 55 and 65 wt % cationic or ionizable lipid.

In some embodiments, the lipid structure may comprise a sterol, such as cholesterol. The lipid structure may comprise at least 1 wt %, at least 5 wt %, at least 10 wt %, at least 15 wt %, at least 20 wt %, at least 25 wt %, at least 30 wt %, at least 35 wt % or at least 40 wt % sterol. The lipid structure may comprise less than 99 wt % sterol, less than 90 wt % sterol, less than 80 wt % sterol, less than 70 wt % sterol, less than 60 wt % sterol, less than 50 wt % sterol or less than 45 wt % sterol. The lipid structure may comprise between 1 and 99 wt % sterol, between 10 and 90 wt % sterol, between 15 and 80 wt % sterol, between 20 and 70 wt % sterol, between 25 and 60 wt % sterol, between 30 and 50 wt % cholesterol or between 35 and 45 wt % sterol.

In some embodiments, the lipid structure may comprise a combination of a cationic or ionizable lipid, such as DOTAP, and a sterol, such as cholesterol. The weight ratio of the cationic or ionizable lipid to the sterol may be between 1:99 and 99:1, between 90 and 90:10, between 20:80 and 85:15, between 30:70 and 80:20, between 40:60 and 75:22, between 50:50 and 70:30 or between 55:45 and 65:35. The weight ratio of the cationic or ionizable lipid to the sterol may be about 60:40.

The sub-micron particle may have an N/P molar ratio of at least 1:50, at least 1:20, at least 1:10, at least 1:5, at least 1:2, at least 1:1, at least 2:1, at least 3:1, at least 4:1 or at least 5:1, more preferably at least 6:1, at least 8:1 or at least 10:1, and most preferably at least 11:1, at least 13:1, at least 15:1, at least 16:1, at least 17:1 or at least 18:1. The sub-micron particle may have an N/P molar ratio of less than 1000:1, less than 500:1, less than 250:1, less than 100:1, less than 50:1, less than 40:1, less than 30:1, less than 28:1, less than 26:1, less than 24:1, less than 22:1, less than 21:1 or less than 20:1. The sub-micron particle may have an N/P molar ratio of between 1:50 and 1,000:1, between 1:10 and 500:1, between 1:5 and 250:1, between 1:2 and 100:1, between 1:1 and 50:1, between 2:1 and 40:1, between 5:1 and 30:1, between 8:1 and 28:1, between 10:1 and 26:1, between 12:1 and 24:1, between 14:1 and 22:1, between 16:1 and 20:1, or between 17:1 and 19:1. In preferred embodiment, the sub-micron particle has an N/P ratio of between 11:1 and 18:1. The N/P molar ratio may be understood to be the ratio between cationic amines in the lipid structure and anionic phosphates in the payload molecule.

The amphiphilic copolymer is preferably an amphiphilic block copolymer.

It may be appreciated that the amphiphilic copolymer may comprise at least one hydrophilic portion and at least one hydrophobic portion. In some embodiment, the amphiphilic copolymer comprises or consists of one hydrophilic portion and one hydrophobic portion. The or each hydrophilic portion may comprise or be a polyether, an amino acid based polymer or polypeptide, poly(2-methyloxazoline) (PMOXA), and/or a derivative thereof. The or each hydrophobic portion may comprise or be a polyester, an acid-labile polycarbonate, poly(ethylethylene) (PEE), poly(butadiene) (PBD), poly(dimethylsiloxane) (PDMS), poly(methyl methacrylate) (PMMA), poly(styrene) (PSt) and/or a derivative thereof.

Preferably, the amphiphilic copolymer is biodegradable. Accordingly, the or each hydrophobic portion may comprise or be a polyester, an acid-labile polycarbonate and/or a derivative thereof.

The acid-labile polycarbonate may be poly(trimethylene carbonate) (PTMC), poly(2,4,6-trimethoxybenzylidenepentaerythritol carbonate) (PTMBPEC) or a derivative thereof. The amino acid based polymer or polypeptide may be poly(L-glutamic acid) (PGA), poly-L-lysine (PLL) or a derivative thereof.

Any suitable polyether may be used. In some embodiments, the or each polyether may be polyethylene glycol (PEG), oligo(ethylene glycol) (oligoEG) or a derivative thereof. For instance, derivatives of PEG could include poly(ethylene glycol) methyl ether acrylate (mPEGA) and poly(ethylene glycol) methyl ether methacrylate (mPEGMA).

Alternatively, the polyether may be a polyether disclosed in the applicant's earlier patent application GB2009720.o. For instance, the hydrophilic portion may comprise or consist of:

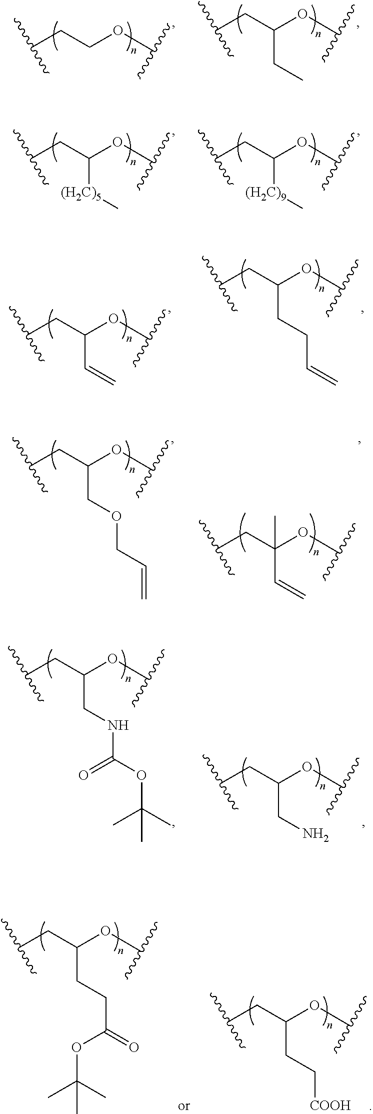

appreciated that n is an integer.

Similarly, any suitable polyester may be used. In some embodiments, the or each polyester may be selected from the list consisting of polycaprolactone (PCL), polylactic acid (PLA), polyglycolide (PGA), poly(lactic-co-glyeolic acid) (PLGA), poly(ε-decalactone) (PDL) or a derivative thereof. Alternatively, the polyester may be a polyester disclosed in the applicant's earlier patent application GB2009720.0. For instance, the hydrophobic portion may comprise or consist of:

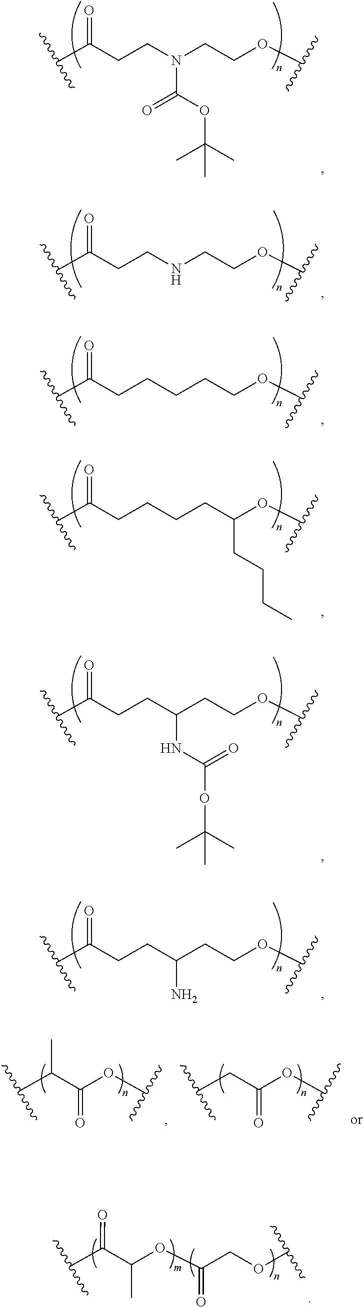

It may be appreciated that m and n are integers.

The hydrophilic portion may comprise less than 60 wt %, less than 50 wt %, less than 45 wt %, less than 40 wt %, less than 35 wt % or less than 32 wt % of the amphiphilic copolymer. The hydrophilic portion may comprise between 5 and 60 wt % of the amphiphilic copolymer, more preferably between 10 and 50 wt % or between 20 and 45 wt % of the amphiphilic copolymer, and most preferably between 25 and 40 wt %, between 28 and 35 wt % or between 30 and 32 wt % of the amphiphilic copolymer.

The hydrophobic portion may comprise at least 40 wt %, at least 50 wt %, at least 55 wt %, at least 60 wt %, at least 65 wt % or at least 68 wt % of the amphiphilic copolymer.

The hydrophobic portion may comprise between 40 and 95 wt % of the amphiphilic copolymer, more preferably between 50 and 90 wt % or between 55 and 80 wt % of the amphiphilic copolymer, and most preferably between 60 and 75 wt %, between 65 and 72 wt % or between 68 and 70 wt % of the amphiphilic copolymer.

The amphiphilic copolymer may have a molecular weight of at least 1,000 Da, at least 2,000 Da, at least 3,000 Da, at least 4,000 Da or at least 5,000 Da. Preferably, the amphiphilic copolymer has a molecular weight of at least 6,000 Da or at least 7,000 Da. In some embodiments, the amphiphilic copolymer has a molecular weight of at least 8,000 Da, at least 10,000 Da, at least 12,000 Da, at least 14,000 Da, at least 15,000 Da or at least 16,000 Da.

The amphiphilic copolymer may have a molecular weight of less than 100,000 Da, less than 80,000 Da, less than 70,000 Da, less than 60,000 Da or less than 50,000 Da. Preferably, the amphiphilic copolymer has a molecular weight of less than 40,000 Da or less than 30,000 Da. Most preferably, the amphiphilic copolymer may have a molecular weight of less than 25,000 Da, less than 20,000 Da, less than 18,000 Da, less than 17,000 Da or less than 16,000 Da.

The amphiphilic copolymer may have a molecular weight of between 1,000 and 100,000 Da, between 2,000 and 80,000, between 3,000 and 70,000, between 4,000 and 60,000, between 5,000 and 50,000, more preferably between 6,000 and 40,000, between 7,000 and 50,000, between 8,000 and 25,000, between 10,000 and 20,000, between 12,000 and 18,000, between 14,000 and 16,000 Da, between 15,000 and 17,500 Da or between 16,000 and 17,000 Da.

The hydrophobic portion may have a molecular weight of at least 1,000 Da, at least 2,000 Da, at least 3,000 Da, at least 4,000 Da or at least 5,000 Da. Preferably, the hydrophobic portion has a molecular weight of at least 6,000 Da or at least 7,000 Da. In some embodiments, the hydrophobic portion has a molecular weight of at least 8,000 Da, at least 10,000 Da or at least 11,000 Da.

The hydrophobic portion may have a molecular weight of between 1,000 and 70,000, between 2,000 and 60,000, between 3000 and 50,000, more preferably between 4,000 and 40,000, between 5,000 and 50,000, between 6,000 and 25,000, between 7,000 and 20,000, between 8,000 and 18,000, between 9,000 and 15,000 Da, between 10,000 and 13,000 Da or between 11,000 and 12,000 Da.

The molecular weight of the amphiphilic copolymer defined above may be understood to be the number-average molecular weight (M.).

The molecular weight of the amphiphilic copolymer, the molecular weight of the hydrophobic portion and/or the molecular weight of the hydrophilic portion may be determined using NMR or gel permeation chromatography (GPC). The methods of using NMR and GPC may be as described in the examples. In some embodiments, the molecular weight is determined using NMR, preferably ¹H NMR.

The weight ratio of the amphiphilic copolymer to the payload molecule may be at least 5:1, at least 10:1, at least 20:1, at least 30:1 or at least 40:1, more preferably at least 50:1, or at least 55:1, and most preferably at least 60:1. The weight ratio of the amphiphilic copolymer to the payload molecule may be less than 1000:1, less than 500:1, less than 250:1, less than 200:1 or less than 150:1, more preferably less than 125:1, less than 100:1, and most preferably less than 85:1. The weight ratio of the amphiphilic copolymer to the payload molecule may be between 5:1 and 1000:1, between 10:1 and 500:1, between 20:1 and 250:1, between 30:1 and 200:1 or between 40:1 and 150:1, more preferably between 50:1 and 125:1, or between 55:1 and 100:1, and most preferably between 60:1 and 85:1.

The weight ratio of the amphiphilic copolymer to the cationic or ionizable lipid may be at least 1:10, at least 1:8, at least 1:6, at least 1:4, at least 1:2 or at least 1:1.5, more preferably at least 1:1, at least 1.5:1, at least 1.75:1, at least 2:1, at least 2.2:1 or at least 2.4:1, and most preferably at least 2.5:1. The weight ratio of the amphiphilic copolymer to the cationic or ionizable lipid may be less than 50:1, less than 20:1, less than 15:1, less than 10:1, less than 8:1 or less than 6:1, more preferably less than 5.5:1, less than 5:1, less than 4.5:1, less than 4:1, less than 3.5:1 or less than 3:1, and most preferably less than 2.7:1. The weight ratio of the amphiphilic copolymer to the cationic or ionizable lipid may be between 1:10 and 50:1, between 1:8 and 20:1, between 1:6 and 15:1, between 1:4 and 10:1, between 1:2 and 8:1 or between 1:1.5 and 6:1, more preferably between 1:1 and 5.5:1, between 1.5:1 and 5:1, between 1.75:1 and 4.5:1, between 2:1 and 4:1, between 2.2:1 and 3.5:1 or between 2.4:1 and 3:1, and most preferably between 2.5:1 and 2.7:1.

The outer layer comprising the amphiphilic copolymer preferably comprises a thickness of at least 0.5 nm, at least 1 nm or at least 1.5 nm, more preferably at least 2 nm or at least 2.5 nm, and most preferably at least 3 nm. The outer layer comprising the amphiphilic copolymer preferably comprises a thickness of less than 25 nm, less than nm or less than 15 nm, more preferably less than 10 nm or less than 7.5 nm, and most preferably less than 5 nm. The outer layer comprising the amphiphilic copolymer preferably comprises a thickness of between 0.5 to 25 nm, between 1 to 20 nm or between 1.5 to 15 nm, more preferably between 2 to 10 nm or between 2.5 to 7.5 nm, and most preferably between 3 to 5 nm.

The sub-micron particle may further comprise at least one stabilizing molecule. The at least one stabilizing molecule may be surrounded by the outer layer comprising the amphiphilic copolymer. Alternatively, or additionally, the at least one stabilizing molecule may be disposed outside the outer layer comprising the amphiphilic copolymer.

The weight ratio of the stabilizing molecule to the payload molecule may be at least 1:1, at least 2:1, at least 4:1, at least 6:1, at least 8:1, at least 10:1, at least 20:1, at least 30:1, at least 40:1, at least 50:1, at least 60:1, at least 70:1, at least 80:1, at least 90:1 or at least 95:1. In some embodiments, the weight ratio of the stabilizing molecule to the payload molecule may be at least 100:1, at least 250:1, at least 500:1, at least 1,000:1, at least 2,000:1, at least 3,000:1, at least 4,000:1 or at least 5,000:1. The weight ratio of the stabilizing molecule to the payload molecule may be less than 100,000:1, less than 50,000:1, less than 10,000:1, less than 9,000:1, less than 8,000 to 1, less than 7,000:1, or less than 6,500:1. In some embodiments, the weight ratio of the stabilizing molecule to the payload molecule may be less than 5,000:1, less than 1,000:1, less than 500:1, less than 400:1, less than 350:1, less than 300:1, less than 250:1, less than 200:1, less than 175:1, less than 150:1, less than 125:1 or less than 110:1. The weight ratio of the stabilizing molecule to the payload molecule may be between 1:1 and 100,000:1, between 2:1 and 50,000:1, between 4:1 and 10,000:1, between 6:1 and 9,000:1, between 8:1 and 8,000:1, between 10:1 and 7,000:1 or between 20:1 and 6,500:1. In some embodiments, the weight ratio of the stabilizing molecule to the payload molecule may be between 6:1 and 5,000:1, between 8:1 and 1,000:1, between 10:1 and 500:1, between 20:1 and 400:1, between 30:1 and 350:1, between 40:1 and 300:1, between 50:1 and 250:1, between 60:1 and 200:1, between 70:1 and 175:1, between 80:1 and 150:1, between 90:1 and 125:1 or between 95:1 and 110:1. In alternative embodiments, the weight ratio of the stabilizing molecule to the payload molecule may be between 90:1 and 100,000:1, between 100:1 and 50,000:1, between 500:1 and 25,000:1, between 1,000:1 and 10,000:1, between 2,000:1 and 9,000:1, between 3,000:1 and 8,000:1, between 4,000:1 and 7,000:1 or between 5,000:1 and 6,500:1. These weight ratios may relate to the total weight of the stabilizing molecule to the payload molecule, i.e. they may include the weight of any stabilizing molecules which are surrounded by the outer layer and any stabilizing molecules which are disposed outside the outer layer.

In embodiments where the at least one stabilizing molecule is surrounded by the outer layer comprising the amphiphilic copolymer may be encapsulated in the lipid structure. Alternatively, or additionally, the at least one stabilizing molecule surrounded by the outer layer comprising the amphiphilic copolymer may be disposed outside the lipid structure. In embodiments where the sub-micron particle comprises a plurality of lipid structures, at least one stabilizing molecule may be disposed between the plurality of lipid structures.

The weight ratio of the stabilizing molecule surrounded by the outer layer to the payload molecule may be at least 1:1, at least 2:1, at least 4:1, at least 6:1, at least 8:1, at least 10:1, at least 20:1, at least 30:1, at least 40:1, at least 50:1, at least 60:1, at least 70:1, at least 80:1, at least 90:1 or at least 95:1. The weight ratio of the stabilizing molecule surrounded by the outer layer to the payload molecule may be less than 100,000:1, less than 50,000:1, less than 10,000:1, less than 5,000:1, less than 1,000:1, less than 500:1, less than 400:1, less than 350:1, less than 300:1, less than 250:1, less than 200:1, less than 175:1, less than 150:1, less than 125:1 or less than 110:1. The weight ratio of the stabilizing molecule surrounded by the outer layer to the payload molecule may be between 1:1 and 100,000:1, between 2:1 and 50,000:1, between 4:1 and 10,000:1, between 6:1 and 5,000:1, between 8:1 and 1,000:1, between 10:1 and 500:1, between 20:1 and 400:1, between 30:1 and 350:1, between 40:1 and 300:1, between 50:1 and 250:1, between 60:1 and 200:1, between 70:1 and 175:1, between 80:1 and 150:1, between go:1 and 125:1 or between 9s:1 and 110:1.

Alternatively or additionally, at least one stabilizing molecule may be disposed outside the outer layer comprising the amphiphilic copolymer. The at least one stabilizing molecule disposed outside the outer layer comprising the amphiphilic copolymer may be disposed at a concentration of at least 1 mg/ml, at least 5 mg/ml, at least 10 mg/ml or at least 50 mg/ml, more preferably at least 100 mg/ml, at least 150 mg/ml, at least 200 mg/ml or at least 220 mg/ml, and most preferably at least 240 mg/ml. The at least one stabilizing molecule disposed outside the outer layer comprising the amphiphilic copolymer may be disposed at a concentration of less than 100,000 mg/ml, less than 50,000 mg/ml, less than 10,000 mg/ml, less than 5,000 mg/ml or less than 1,000 mg/ml, more preferably less than 750 mg/ml, less than 500 mg/ml, less than 300 mg/ml or less than 280 mg/ml, and most preferably less than 260 mg/ml. The at least one stabilizing molecule disposed outside the outer layer comprising the amphiphilic copolymer may be disposed at a concentration of between 1 and 100,000 mg/ml, between 5 and 50,000 mg/ml, between 10 and 10,000 mg/ml, between 25 and 5,000 mg/ml or between 50 and 1,000 mg/ml, more preferably between 100 and 750 mg/ml, between 150 and 500 nm/ml, between 200 and 300 nm/ml, or between 220 and 280 nm/ml, most preferably between 240 and 260 mg/ml.

In a preferred embodiment, the sub-micron particle comprises at least one stabilizing molecule surrounded by the outer layer comprising the amphiphilic copolymer and at least one stabilizing molecule is disposed outside the outer layer comprising the amphiphilic copolymer.

The or each stabilizing molecules may be a carbohydrate and/or a polyol.

The carbohydrate may be referred to as a sugar. The carbohydrate may be a monosaccharide, which may be selected from a group consisting of: glucose; galactose; fructose; mannose; and xylose or a pharmaceutically acceptable complex, salt, solvate, tautomeric form, stereoisomer or polymorphic form thereof. Alternatively, the carbohydrate may be a disaccharide, which may be selected from a group consisting of: trehalose; sucrose; lactose; maltose; isomaltose; lactitol; lactulose; mannobiose; and isomalt or a pharmaceutically acceptable complex, salt, solvate, tautomeric form, stereoisomer or polymorphic form thereof. In a further alternative, the carbohydrate may be a trisaccharide, which may be selected from a group consisting of: nigerotriose; maltotriose; melezitose; maltotriulose; raffinose; and kestose or a pharmaceutically acceptable complex, salt, solvate, tautomeric form, stereoisomer or polymorphic form thereof. In a further alternative, the carbohydrate may be a polysaccharide, which may be selected from the group consisting of: dextran; amylose; amylopectin; glycogen; galactogen; inulin; callose; cellulose; chitosan; and chitin or a pharmaceutically acceptable complex, salt, solvate, tautomeric form, stereoisomer or polymorphic form thereof.

The carbohydrate may be a polyol, which may be selected from a group consisting of: sorbitol; mannitol; glycerol; alpha-D-glucopyranosyl-1-6-sorbitol; alpha-D-glucopyranosyl-1-6-mannitol; a malto-oligosaccharide; a hydrogenated maltooligosaccharide, starch and cellulose or a pharmaceutically acceptable complex, salt, solvate, tautomeric form, stereoisomer or polymorphic form thereof.

Alternatively or additionally, the polyol may be an oligomer comprising a plurality of hydroxyl groups; a polymer comprising a plurality of hydroxyl groups or a pharmaceutically acceptable complex, salt, solvate, tautomeric form, stereoisomer or polymorphic form thereof.

The at least one stabilizing molecule may comprise at least two different stabilizing molecules. Each stabilizing molecule may be a carbohydrate. In one embodiment, a first stabilizing molecule may be a disaccharide (e.g., trehalose) and a second stabilizing molecule may be a polysaccharide (e.g., dextran).

In some embodiments, the carbohydrate is a disaccharide, and most preferably trehalose, or a pharmaceutically acceptable complex, salt, solvate, tautomeric form, stereoisomer or polymorphic form thereof. The trehalose may be synthetic trehalose or natural trehalose.

The sub-micron particle may comprise at least one targeting ligand or moiety. The at least one targeting ligand or moiety may be disposed on an outer surface of the sub-micron particle. Accordingly, the at least one targeting ligand or moiety may be disposed on an outer surface of the outer layer comprising the amphiphilic copolymer.

The at least one targeting ligand or moiety may be or comprise at least one of a peptide, a protein, an aptamer, a carbohydrate, an oligosaccharide, a folic acid or folate, and antibody or an antigen binding fragment thereof, a vitamin or a derivative thereof. The peptide may be a G protein-coupled receptor (GCR), Arg-Gly-Asp (RGD), or a derivative thereof. The proteins may be a lectin, a transferrin, or a derivative thereof.

The aptamers may be an RNA aptamer against HIV glycoprotein, or a derivative thereof. The carbohydrates may be as defined above. In particular, the carbohydrate may be mannose, glucose, galactose, or a derivative thereof. The antibody may be monoclonal or polyclonal. The antibody may be an anti-Her2 antibody, an anti-EGFR antibody, or a derivative thereof. The vitamin may be vitamin D.

The sub-micron particle may be stored in solution. The solution may be an aqueous solution. The sub-micron particle may be present at a concentration such that the concentration of the payload molecule is at least 0.001 μg/ml, at least 0.01 μg/ml, at least 0.05 μg/ml, at least 0.1 μg/ml, at least 0.5 μg/ml, at least 1 μg/ml, at least 5 μg/ml, at least 10 μg/ml, at least 15 μg/ml or at least 20 μg/ml. The sub-micron particle may be present at a concentration such that the concentration of the payload molecule is less than 500 mg/ml, less than 100 mg/ml, less than 10 mg/ml, less than 5 mg/ml, less than 1 mg/ml, less than 500 μg/ml, less than 200 μg/ml, less than 100 μg/ml, less than 50 μg/ml or less than 30 μg/ml. The sub-micron particle may be present at a concentration such that the concentration of the payload molecule is between 0.001 μg/ml and 500 mg/ml, between 0.01 μg/ml and 100 mg/ml, between 0.05 μg/ml and 50 mg/ml, between 0.1 μg/ml and 10 mg/ml, between 0.5 μg/ml and 5 mg/ml, between 1 μg/ml and 1 mg/ml, between 5 and 500 μg/ml, between 10 and 200 μg/ml, between and 100 μg/ml, between 20 and 50 μg/ml or between 20 and 30 μg/ml.

Alternatively, the sub-micron particle may be freeze dried.

Preferably, the sub-micron particle of the first aspect is thermally stabilized.

It will be appreciated that the expression “thermal stabilization” or “thermally stabilized” can mean that the sub-micron particle substantially retains its biological activity (e.g., it elicits an immune response and/or protein expression in a subject administered therewith) when stored at certain temperatures for a period of time. Although the inventors do not wish to be bound by any hypothesis, they believe that the thermal stabilization effects may be realised by stabilizing the lipid structure in the formulation, for example by preventing or reducing its aggregation; by stabilizing the payload molecule (preferably RNA) per se; and/or by stabilizing the sub-micron particle to have improved colloidal stability. Whether or not the functional activity of the payload molecule (preferably RNA) is retained, or the extent thereof, can be determined for example by detecting the presence of immunospecific antibodies (e.g., IgG) raised against the antigen of interest encoded by the RNA construct and/or detecting the expression of a protein of interest.

Preferably, the sub-micron particle is thermally stabilised following storage at a temperature of −100° C. and above, −80° C. and above, −60° C. and above, −40° C. and above or −20° C. and above, more preferably −15° C. and above, and most preferably −10° C. and above. Preferably, the sub-micron particle is thermally stabilised following storage at a temperature of −5° C. and above, more preferably ° C. and above, and most preferably 1C and above. Most preferably, the sub-micron particle is thermally stabilised following storage at a temperature of 2° C. and above, more preferably 3° C. and above, and most preferably 4° C. and above. Even more preferably, the sub-micron particle is thermally stabilised following storage at a temperature of 5° C. and above, more preferably 6° C. and above, and most preferably 7° C. and above.

The sub-micron particle may be thermally stabilised following storage at a temperature of less than 100° C., less than 80° C., less than 60° C., less than 50° C., less than 40° C., less than 35° C., or less than 30° C. The sub-micron particle may be thermally stabilised following storage at a temperature of less than 25° C., less than 20° C., or less than 15° C. The sub-micron particle may be thermally stabilised following storage at a temperature of less than 100° C., less than 8° C., or less than 7° C.

The sub-micron particle may be thermally stabilised following storage at a temperature of between −100° C. and 100° C., between −80° C. and 90° C., between −60° C. and 80° C., between −40° C. and 70° C., between −20° C. and 60° C., between −20° C. and 50° C., between −20° C. and 40° C., between −20° C. and 35° C., between −2° C. and 30° C., between −15° C. and 25° C., or between −10° C. and 20° C. The sub-micron particle may be thermally stabilised following storage at a temperature of between −5° C. and 15° C., between 0° C. and 10° C., between 1° C. and 9° C., or between 2° C. and 8° C.

In accordance with a second aspect, there is provided a method of producing a sub-micron particle, the method comprising contacting a payload molecule, a cationic or ionizable lipid, and an amphiphilic copolymer to produce the sub-micron particle.

Advantageously, the method provides a one-pot method for providing the sub-micron particle of the first aspect.

Preferably, the payload molecule, the cationic or ionizable lipid, and the amphiphilic copolymer are contacted simultaneously. The payload molecule, cationic or ionizable lipid and amphiphilic copolymer may be understood to be contacted simultaneously if they are all present in the same reaction mixture.

The payload molecule, the cationic or ionizable lipid and the amphiphilic copolymer may be as defined in relation to the first aspect. Furthermore, the payload molecule, the cationic or ionizable lipid and the amphiphilic copolymer may be provided at the ratios defined in relation to the first aspect.

The amphiphilic copolymer may be synthesized using any method known in the art. For instance, the amphiphilic copolymer may be synthesized using the method defined in the applicant's earlier patent application, GB2009720.0. However, it will be appreciated that alternative methods may be used.

The method may comprise providing a first solution comprising the cationic or ionizable lipid and the amphiphilic copolymer. The first solution may comprise an organic solvent. The organic solvent may be an ether, an alcohol or a nitrile. The ether may be a cyclic ether. The organic solvent may be tetrahydrofuran (THF), ethanol, methanol and acetronitrile.

The method may comprise providing a second solution comprising the payload molecule. The second solution may comprise water, preferably ribonuclease (RNase)-free water.

Contacting the payload molecule, the cationic or ionizable lipid and the amphiphilic copolymer may comprise combining the first and second solutions to produce a reaction mixture, and thereby contacting the payload molecule, the cationic or ionizable lipid, and the amphiphilic copolymer.

The method may comprise stirring the reaction mixture.

The method may comprise contacting the payload molecule, the cationic or ionizable lipid and the amphiphilic copolymer for at least 15 seconds, at least 30 seconds, at least seconds or at least 1 minute. The method may comprise contacting the payload molecule, the cationic or ionizable lipid and the amphiphilic copolymer for between 15 seconds and 30 minutes, between 30 seconds and 10 minutes, between 45 seconds and 5 minutes or between 1 and 2 minutes.

The method may comprise contacting the payload molecule, the cationic or ionizable lipid and the amphiphilic copolymer at a temperature between ° C. and 75° C., between 5° C. and 50° C., between 10° C. and 30° C. between 15° C. and 25° C. or between 19° C. and 21° C.

The method may subsequently comprise removing the organic solvent. The organic solvent may be removed by rotary evaporation.

Contacting the payload molecule, the cationic or ionizable lipid and the amphiphilic copolymer, may comprise contacting the payload molecule, the cationic or ionizable lipid, the amphiphilic copolymer and a stabilizing molecule. The payload molecule, the cationic or ionizable lipid, the amphiphilic copolymer and the stabilizing molecule may be contacted simultaneously.

Advantageously, in the resultant sub-micron particle the stabilizing molecule will be surrounded the outer layer comprising the amphiphilic copolymer.

The stabilizing molecule may be as defined in relation to the first aspect.

In embodiments where the method comprises providing a second solution, the second solution may further comprise the stabilizing molecule.

The weight ratio of the stabilizing molecule to the payload molecule in the second solution and/or in the reaction mixture may be at least 1:1, at least 2:1, at least 4:1, at least 6:1, at least 8:1, at least 10:1, at least 20:1, at least 30:1, at least 40:1, at least 50:1, at least 60:1, at least 70:1, at least 80:1, at least 90:1 or at least 95:1. The weight ratio of the stabilizing molecule to the payload molecule in the second solution and/or in the reaction mixture may be less than 100,000:1, less than 50,000:1, less than 10,000:1, less than 5,000:1, less than 1,000:1, less than 500:1, less than 400:1, less than 350:1, less than 300:1, less than 250:1, less than 200:1, less than 175:1, less than 150:1, less than 125:1 or less than 110:1. The weight ratio of the stabilizing molecule to the payload molecule in the second solution and/or in the reaction mixture may be between 1:1 and 100,000:1, between 2:1 and 50,000:1, between 4:1 and 10,000:1, between 6:1 and 5,000:1, between 8:1 and 1,000:1, between 10:1 and 500:1, between 20:1 and 400:1, between 30:1 and 350:1, between 40:1 and 300:1, between 50:1 and 250:1, between 60:1 and 200:1, between 70:1 and 175:1, between 80:1 and 150:1, between 90:1 and 125:1 or between 95:1 and 110:1.

Alternatively, or additionally, the method may comprise contacting the resultant sub-micron particle and a stabilizing molecule. The stabilizing molecule may be as defined in relation to the first aspect. Accordingly, at least one stabilizing molecule may be disposed outside the outer layer comprising the amphiphilic copolymer.

The method may comprise contacting the resultant sub-micron particle and the stabilizing molecule subsequent to removing the organic solvent. The method may comprise contacting the resultant sub-micron particle and the stabilizing molecule at a concentration to obtain the concentration of the stabilizing molecule defined in relation to the first aspect.

The method may comprise storing the sub-micron particle in solution. The solution may be as defined in relation to the first aspect.

Alternatively, the method may comprise drying the sub-micron particle. Preferably, drying the sub-micron particle comprise freeze drying the sub-micron particle.

In accordance with a third aspect, there is provided a sub-micron particle obtained or obtainable by the method of the second aspect.

In a fourth aspect, there is provided a pharmaceutical composition comprising the sub-micron particle of the first or third aspect and a pharmaceutically acceptable vehicle.

In a fifth aspect, there is provided a method of preparing the pharmaceutical composition according to the fourth aspect, the method comprising contacting the sub-micron particle of the first or third aspect with a pharmaceutically acceptable vehicle.

In a sixth aspect, there is provided the sub-micron particle of the first or third aspect, or the pharmaceutical composition of the fourth aspect, for use as a medicament.

In a seventh aspect, there is provided a method of treatment, the method comprising administering, or having administered, to a subject in need thereof, a therapeutic amount of the sub-micron particle of the first or third aspect, or the pharmaceutical composition of the fourth aspect.

In an eighth aspect, there is provided a vaccine composition comprising the sub-micron particle of the first or third aspect, or the pharmaceutical composition of the fourth aspect.

The vaccine may comprise a suitable adjuvant.

The vaccine may be a vaccine for COVID-19. The vaccine may be a vaccine for influenza virus.

In a ninth aspect, there is provided the sub-micron particle of the first or third aspect, the pharmaceutical composition of the fourth aspect or the vaccine of the eighth aspect, for use in stimulating an immune response in a subject.

The immune response may be stimulated against a protozoa, bacterium, virus, fungus or cancer. The virus may be COVID-19. The virus may be influenza virus.

In a tenth aspect of the invention, there is provided a method of vaccinating a subject, the method comprising administering, or having administered, to a subject in need thereof, a therapeutic amount of the sub-micron particle of the first or third aspect, the pharmaceutical composition of the fourth aspect or the vaccine of the eighth aspect.

The sub-micron particle, the pharmaceutical composition or the vaccine of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

The sub-micron particle, the pharmaceutical composition or the vaccine of the invention may also be incorporated within a slow- or delayed-release device. Such devices may, for example, be inserted on or under the skin, and the medicament may be released over weeks or even months. The device may be located at least adjacent the treatment site.

In a preferred embodiment, however, medicaments according to the invention may be administered to a subject by injection into the blood stream, muscle, skin or directly into a site requiring treatment. Injections may be intravenous (bolus or infusion), subcutaneous (bolus or infusion), intradermal (bolus or infusion), intramuscular (bolus or infusion), intrathecal (bolus or infusion), epidural (bolus or infusion) or intraperitoneal (bolus or infusion).

It will be appreciated that the amount of sub-micron particle, the pharmaceutical composition or the vaccine that is required is determined by its biological activity and bioavailability, which in turn depends on the mode of administration, the physiochemical properties of the sub-micron particle, the pharmaceutical composition or the vaccine and whether it is being used as a monotherapy or in a combined therapy.

The frequency of administration will also be influenced by the half-life of the active agent within the subject being treated. Optimal dosages to be administered may be determined by those skilled in the art, and will vary with the sub-micron particle, the pharmaceutical composition or the vaccine in use, the strength of the pharmaceutical composition, the mode of administration, and the type of treatment. Additional factors depending on the particular subject being treated will result in a need to adjust dosages, including subject age, weight, gender, diet, and time of administration.

The required dose may depend upon a number of factors including, but not limited to, the active agent being administered, the disease being treated and/or vaccinated against, the subject being treated, etc.

Generally, a dose of between 0.001 μg/kg of body weight and 10 mg/kg of body weight, or between 0.01 μg/kg of body weight and 1 mg/kg of body weight, of the sub-micron particle, the pharmaceutical composition or the vaccine of the invention may be used, depending upon the active agent used. A dose may be understood to relate to the quantity of the payload molecule which is delivered.

Doses may be given as a single administration (e.g., a single injection). Alternatively, the sub-micron particle, the pharmaceutical composition or the vaccine may require more than one administration. As an example, the sub-micron particle, the pharmaceutical composition or the vaccine may be administered as two or more doses of between 0.07 μg and 700 mg (i.e., assuming a body weight of 70 kg). Alternatively, a slow-release device may be used to provide optimal doses of the sub-micron particle, the pharmaceutical composition or the vaccine according to the invention to a patient without the need to administer repeated doses. Routes of administration may incorporate intravenous, intradermal subcutaneous, intramuscular, intrathecal, epidural or intraperitoneal routes of injection.

Known procedures, such as those conventionally employed by the pharmaceutical industry (e.g., in vivo experimentation, clinical trials, etc.), may be used to form specific formulations of the sub-micron particle, the pharmaceutical composition or vaccine according to the invention and precise therapeutic regimes (such as doses of the agents and the frequency of administration).

A “subject” may be a vertebrate, mammal, or domestic animal. Hence, compositions and medicaments according to the invention may be used to treat any mammal, for example livestock (e.g., a horse), pets, or may be used in other veterinary applications. Most preferably, however, the subject is a human being.

A “therapeutically effective amount” of the sub-micron particle, the pharmaceutical composition or the vaccine is any amount which, when administered to a subject, is the amount of the aforementioned that is needed to produce a therapeutic effect.

For example, a therapeutically effective amount of the sub-micron particle, the pharmaceutical composition and the vaccine of the invention may comprise from about 0.001 mg to about 800 mg of the payload molecule, and preferably from about 0.01 mg to about 500 mg of the payload molecule.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder, a capsule or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g., sub-micron particle of the invention) may be mixed with a vehicle having the necessary compression properties in suitable proportions and compacted in the shape and size desired. The pharmaceutical vehicle may be a gel and the composition may be in the form of a cream or the like.

Alternatively, the pharmaceutical vehicle may be a liquid, and the pharmaceutical composition is in the form of a solution. Liquid vehicles are used in preparing solutions, suspensions, emulsions, syrups, elixirs and pressurized compositions. The sub-micron particle according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and subcutaneous injection. The sub-micron particle of the invention may be prepared as any appropriate sterile injectable medium.

The sub-micron particle may be administered by inhalation. For instance, the sub-micron particle may be provided in the form of an aerosol.

The sub-micron particle and/or the pharmaceutical composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like.

The sub-micron particle of the invention and/or the pharmaceutical composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All features described herein (including any accompanying claims, abstract and drawings), and/or all of the steps of any method or process so disclosed, may be combined with any of the above aspects in any combination, except combinations where at least some of such features and/or steps are mutually exclusive.

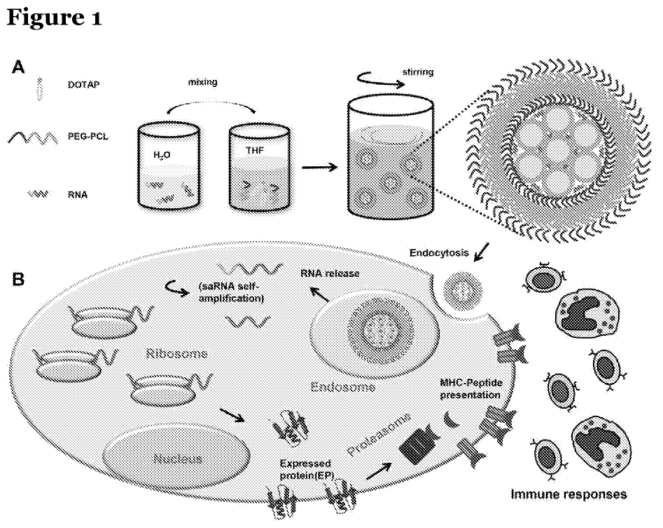

For a better understanding of the invention, and to show how embodiments of the same may be carried into effect, reference will now be made, by way of example, to the accompanying drawings, in which:-FIG. 1 is a schematic illustration of (A) the preparation procedure of polymer-enveloped lipid nanoparticles (PE-LNPs) and (B) the PE-LNP mediated intracellular delivery of RNA including messenger RNA (mRNA) or self-amplifying RNA (saRNA, its self-amplification step depicted in the parentheses). PEG₅ₖ-PCL₁₀ₖ and DOTAP were used to prepare PE-LNPs unless specified;

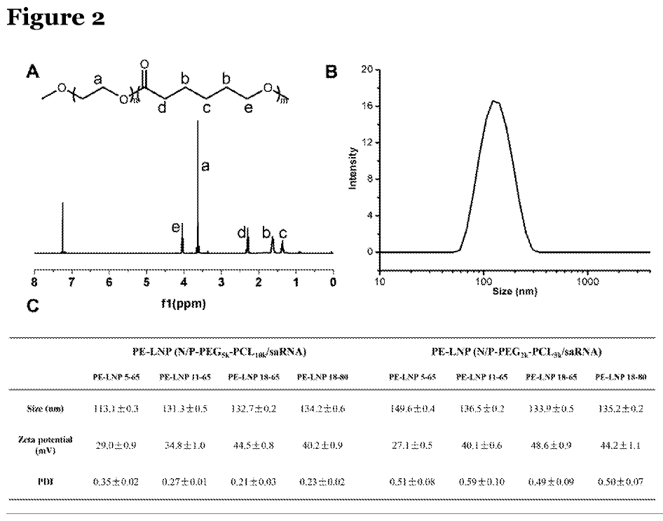

FIG. 2 shows (A)¹H-NMR spectrum of PEG₅ₖ-PCL₁₀ₖ polymer; (B) dynamic light scattering (DLS) size distribution of PE-LNP 18-80 comprised of PEG₅ₖ-PCL_{I}k, DOTAP and saRNA; and (C) DLS size, zeta potential and polydispersity (PDI) of different PE-LNPs;

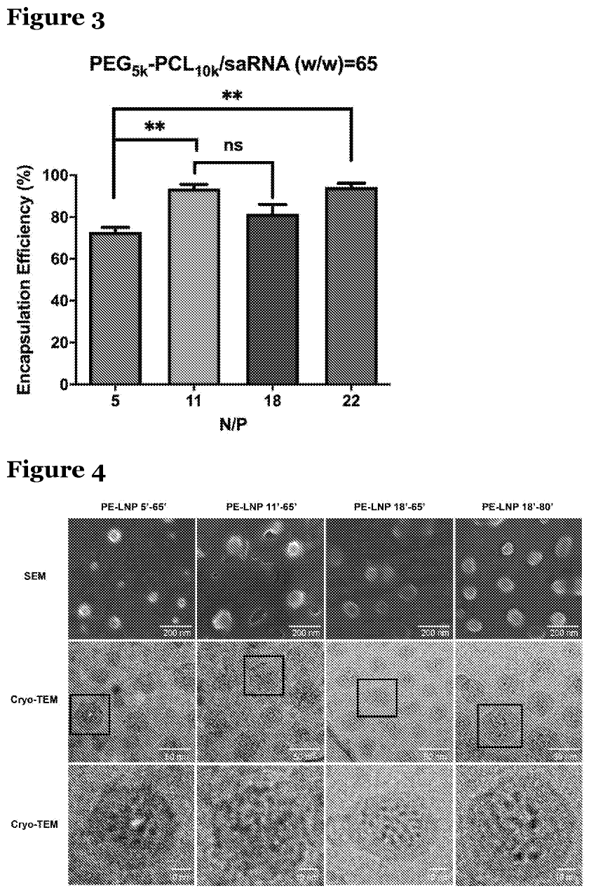

FIG. 3 shows the encapsulation efficiency, determined by RiboGreen Assay, of the PE-LNPs with different N/P molar ratios (PEG₅ₖ-PCL₁₀ₖ/saRNA weight ratio=65);

FIG. 4 shows SEM and Cryo-TEM micrographs of PE-LNP 5′-65′, PE-LNP 11′-65′, PE-LNP 18′-65′ and PE-LNP 18′-80′. The apostrophe symbols suggest the absence of payload in PE-LNPs;

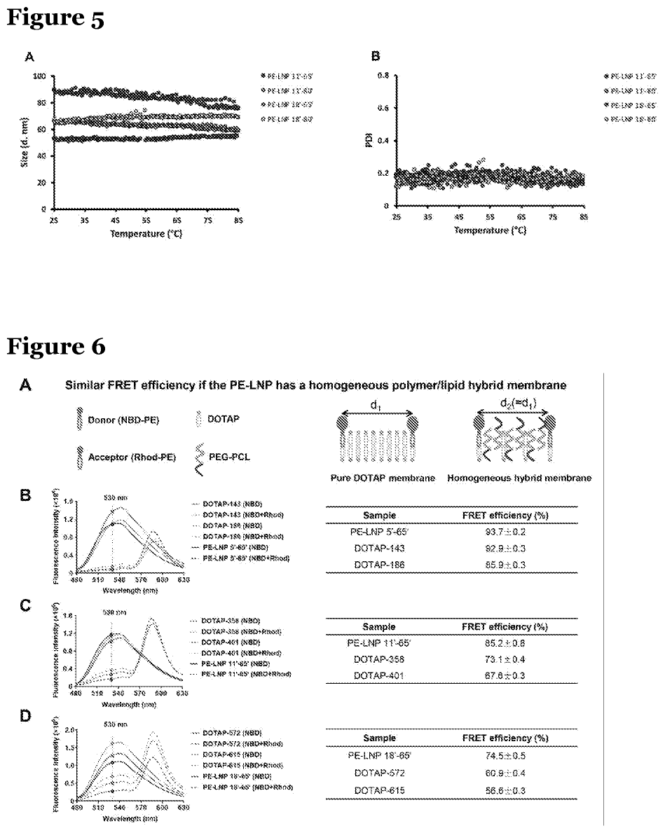

FIG. 5 shows the changes in (A) DLS size and (B) PDI upon thermal ramp of PE-LNP 11′-65′. PE-LNP 11′-80′, PE-LNP 18′-65′ and PE-LNP 18′-80′. The apostrophe symbols suggest the absence of payload in PE-LNPs;

FIG. 6(A) is a schematic illustration of the homogeneous polymer-lipid hybrid membrane which shows the similar distance between NBD (donor dye) and Rhod (acceptor dye) and thus the similar fluorescence resonance energy transfer (FRET) efficiency, compared to the pure lipid membrane; and (B-D) show fluorescence spectra and FRET efficiencies of PE-LNPs (PE-LNP 5′-65′, PE-LNP 11′-65′ and PE-LNP 18′-65′) and DOTAP liposomes. The apostrophe symbols suggest the absence of payload in PE-LNPs. The smaller number after DOTAP in each sample group, i.e., 143, 358 and 572, denotes to equivalent molar concentrations of DOTAP present in the corresponding PE-LNP, respectively. The larger number after DOTAP in each sample group, i.e., 186, 401 and 615, denotes to total molar concentrations of DOTAP and PEG₅ₖ-PCL₁₀ₖ present in the corresponding PE-LNP, respectively. The concentration of NBD-PE (N-(7-Nitrobenz-2-oxa-1,3-diazol-4-yl)-1,2- dihexadecanoyl-snglycero-3-phosphoethanolamine, triethylammonium salt) and Rhod-PE (1,2-dipalmitoyl- sn-glycero-3-phosphoethanolamine-N-(lissamine rhodamine B sulfonyl), ammonium salt) in the PE-LNPs or DOTAP liposomes was fixed at 4.0 mM, and their fluorescence intensities were measured at an excitation wavelength of 460 nm. The significant differences in the FRET efficiency suggests there was no existence of homogeneous polymer-lipid hybrid membrane;

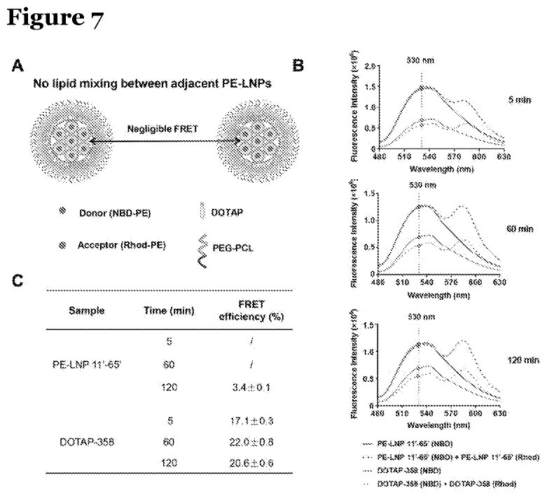

FIG. 7(A) is a schematic illustration of the absence of lipid mixing if DOTAP is present in the core of PE-LNP, resulting negligible FRET; and (B, C) show fluorescence spectra and FRET efficiencies of PE-LNP 11′-65′ and DOTAP liposomes at the 0, 1 and 2 h time intervals. The apostrophe symbols suggest the absence of payload in PE-LNPs. The number after DOTAP, i.e., 358, denotes to equivalent molar concentrations of DOTAP present in PE-LNP 11′-65′. The concentration of NBD-PE and Rhod-PE in PE-LNP 11′-65′ or DOTAP liposomes was fixed at 4.0 mM, and their fluorescence intensities were measured at an excitation wavelength of 460 nm. The negligible FRET after the mixing of PE-LNP 11′-65′ (NBD-PE) and PE-LNP 11′-65′ (Rhod-PE) for 2 h suggests the presence of DOTAP lipids in the core of PE-LNPs;

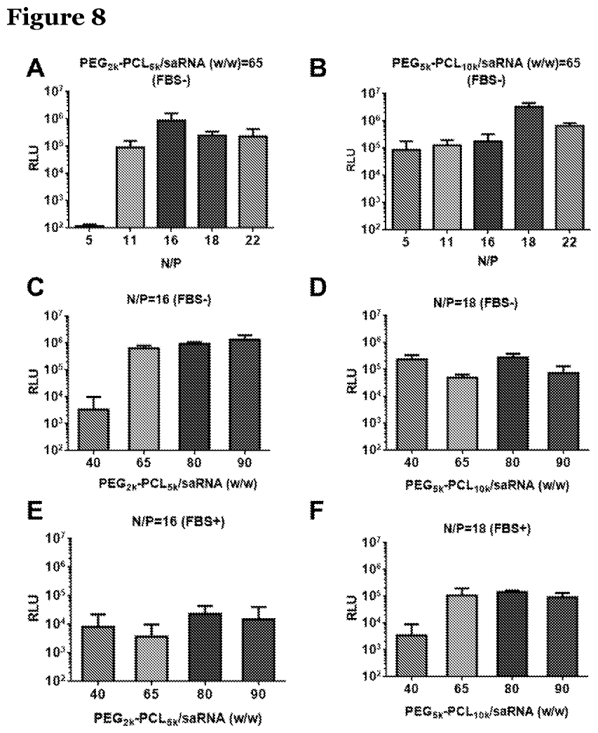

FIG. 8 shows graphs of (A, B) HEK₂₉₃ cell transfection efficiency, expressed as the relative luminescence unit (RLU) determined by Firefly Luciferase (fLuc) assay, of saRNA-loaded PE-LNPs with different N/P molar ratios in the absence of fetal bovine serum (FBS); and HEK₂₉₃ cell transfection efficiency of saRNA-loaded PE-LNPs with different PEG-PCL/saRNA (w/w) ratios (C, D) in the absence of FBS or (E, F) in the presence of FBS. The saRNA dose was fixed at 1 μg mL-¹;

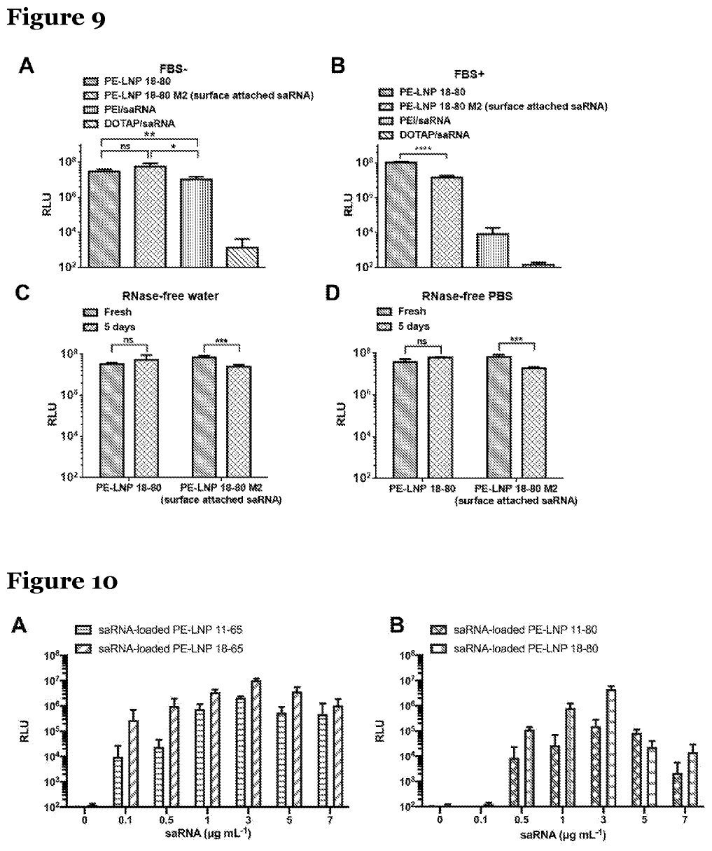

FIG. 9 compares HEK₂₉₃ cell transfection efficiency of the saRNA-loaded PE-LNP 18-80 (saRNA in the nanoparticle core) and the saRNA-attached PE-LNP 18-80 M₂ (saRNA on the nanoparticle surface) (A) in the absence of FBS or (B) in the presence of FBS; and after storage at 4° C. for 5 days (C) in RNase-free water or (D) in RNase-free PBS. The saRNA dose was fixed at 1 μg mL⁻¹. The single (*), double (**), triple (***) and quadruple asterisk symbols (****) denote to p<0.05, p<0.01, p<0.001 and p<0.0001, respectively, and NS represents no significant difference between two groups;

FIG. 10 shows HeLa cell transfection efficiency of the saRNA-loaded PE-LNPs with polymer/saRNA weight ratios of (A) 65 and (B) 80 (N/P molar ratio=11 or 18). The saRNA doses of 0.1, 0.5, 1, 3, 5 and 7 μg mL⁻¹ were tested in the Firefly Luciferase (fLuc) assay;

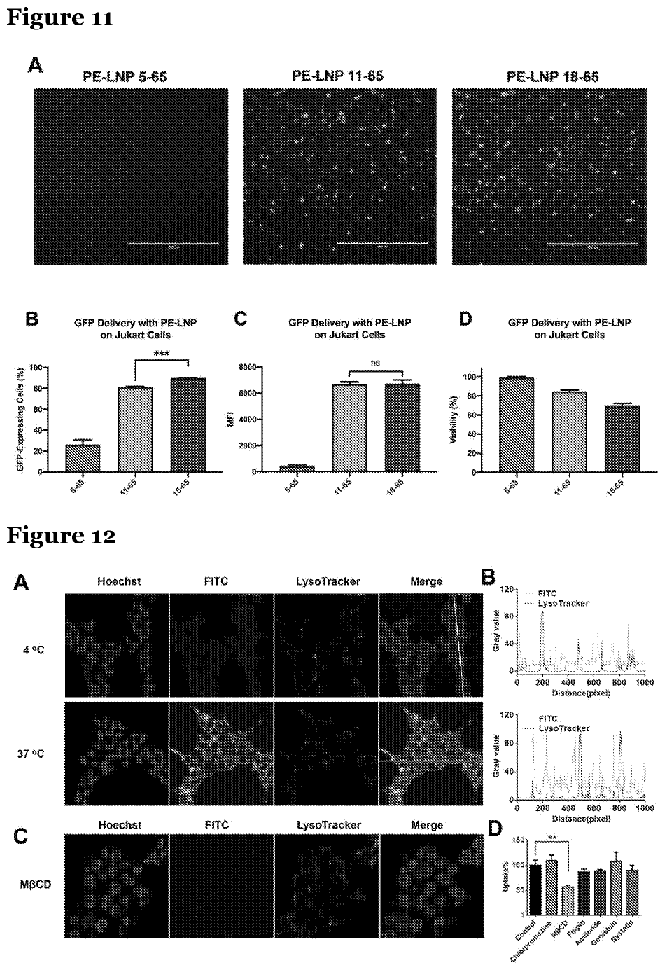

FIG. 11 shows (A) fluorescence microscopy images showing the Jurkat cells transfected with various PE-LNPs loaded with green fluorescent protein (GFP)-encoding messenger RNA (mRNA) in RPMI-1640 medium; (B,C) flow cytometry analysis of the percentage of GFP-expressing Jurkat cells and mean fluorescence intensity (MFI) and (D) cell viability relative to negative control (cells treated with medium only) measured using a Cell Viability Analyzer after treatment of PE-LNPs for 4 h in OPTIMEM medium. The mRNA dose was fixed at 2 μg mL-¹. The single (*) and quadruple asterisk symbols (***) denote to p<0.05 and p<0.0001, respectively, and NS represents no significant difference between two groups;

FIG. 12 shows (A) confocal microscopy images showing the intracellular distribution of PE-LNP 18-80 (coloaded with 1 μg mL⁻¹ saRNA and 50 μg mL⁻¹ FITC) in HEK₂₉₃ cells at 4 or 37° C. for 4 h. Yellow line indicates the cross section used to produce the intensity profiles shown in (B) with the use of ImageJ; (C) confocal microscopy images showing the intracellular distribution of the saRNA- and FITC-coloaded PE-LNP 18-80 in HEK₂₉₃ cells in the presence of an endocytic inhibitor MβCD; and (D) flow cytometry analysis of endocytosis of the saRNA- and FITC-coloaded PE-LNP 18-80 in the presence of various endocytic inhibitors. The double asterisk symbol (**) denotes to p<0.01;

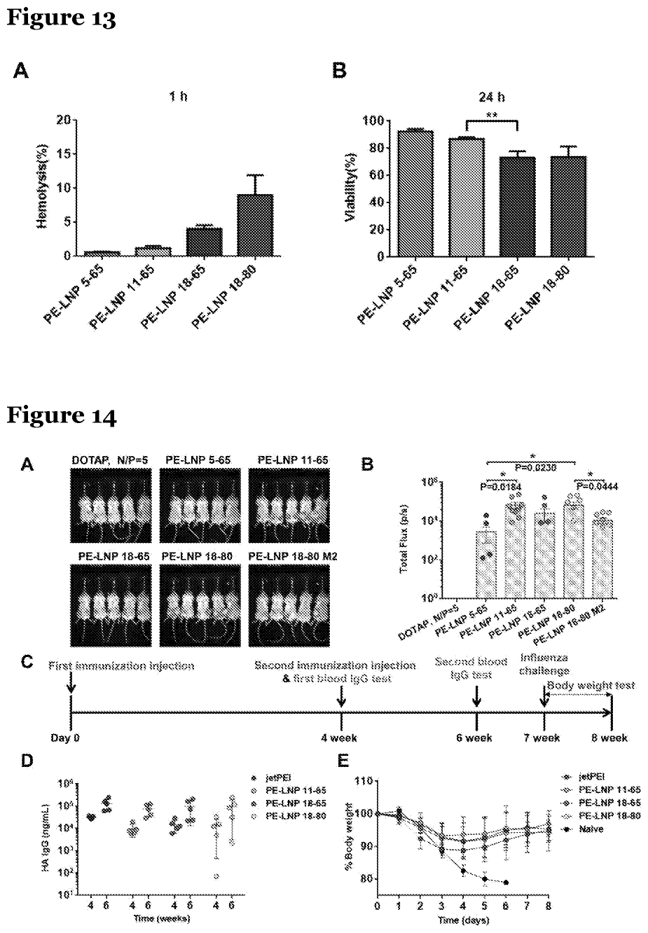

FIG. 13 shows graphs of (A) hemolysis after incubation with various PE-LNPs loaded with 1 μg mL-¹ saRNA at 37° C. for 1 h; and (B) viability relative to negative control (cells treated with medium only) of HEK₂₉₃ cells treated with the saRNA-loaded PE-LNPs for 24 h measured using alamarBlue assay. The double asterisk symbol (**) denotes to p<0.01;

FIG. 14 shows (A) a visualization of fLuc bioluminescence and (B) quantification of fLuc expression in Balb/C female mice on day 7 after intramuscular (IM) injection in both hind leg quadriceps muscles with 5 μg of fLuc saRNA formulated in the interior or on the surface of PE-LNPs; (C) a schematic illustration of the immunogenicity experiments in Balb/C female mice immunized IM in one hind leg quadriceps muscle with 1 μg of saRNA encoding H₁ hemagglutinin of the Cal/09 virus (HA saRNA) formulated in the interior of various PE-LNPs and boosted with the identical formulation after 4 weeks; (D) HA antigen-specific IgG antibody titers following immunization of the mice with prime and boost of HA saRNA-loaded jetPEI and different PE-LNPs via IM injection; and (E) change in body weight after intranasal (IN) challenge with Cal/09 flu virus for Balb/C female mice injected IM. The single asterisk symbol (*) denotes to p<0.05;

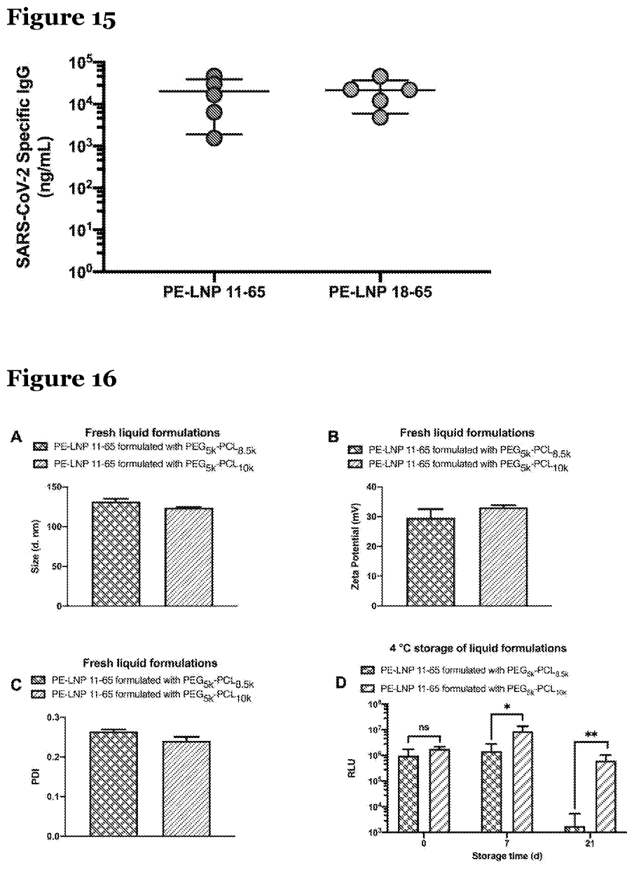

FIG. 15 shows the SARS-CoV-2 (COVID-19)-specific IgG antibody titers following immunization of the mice with prime of SARS-CoV-2 saRNA-loaded PE-LNP 11-65 and PE-LNP 18-65 via IM injection;

FIG. 16 shows the graphs of the saRNA-loaded PE-LNP 11-65 comprised of PEG₅ₖ-PCL₃₋₅ₖ and PEG₅ₖ-PCL₁₀ₖ, respectively: (A) DLS size, (B) PDI and (C) zeta potential of the fresh samples and (D) HEK₂₉₃ cell transfection efficiency of the samples after storage in aqueous solution at 4° C. for 21 days. The saRNA dose was fixed at 1 μg mL-¹;

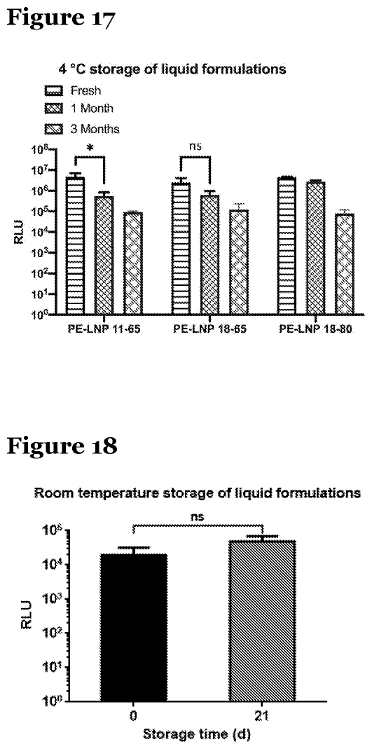

FIG. 17 shows stable storage of 40 μg mL⁻¹ saRNA formulated in PE-LNP 11-65, PE-LNP 18-65 and PE-LNP 18-80, respectively, in aqueous solution at 4° C. This graph shows the HEK₂₉₃ cell transfection efficiency after storage in aqueous solution at 4° C. for three months. The saRNA dose was fixed at 1 μg mL-1;

FIG. 18 shows the HEK₂₉₃ cell transfection efficiency of 40 μg mL⁻¹ saRNA formulated in PE-LNP 11-65 after storage in aqueous solution at room temperature for 21 days. The saRNA dose was fixed at 1 μg mL⁻¹; NS represents no significant difference between two groups;

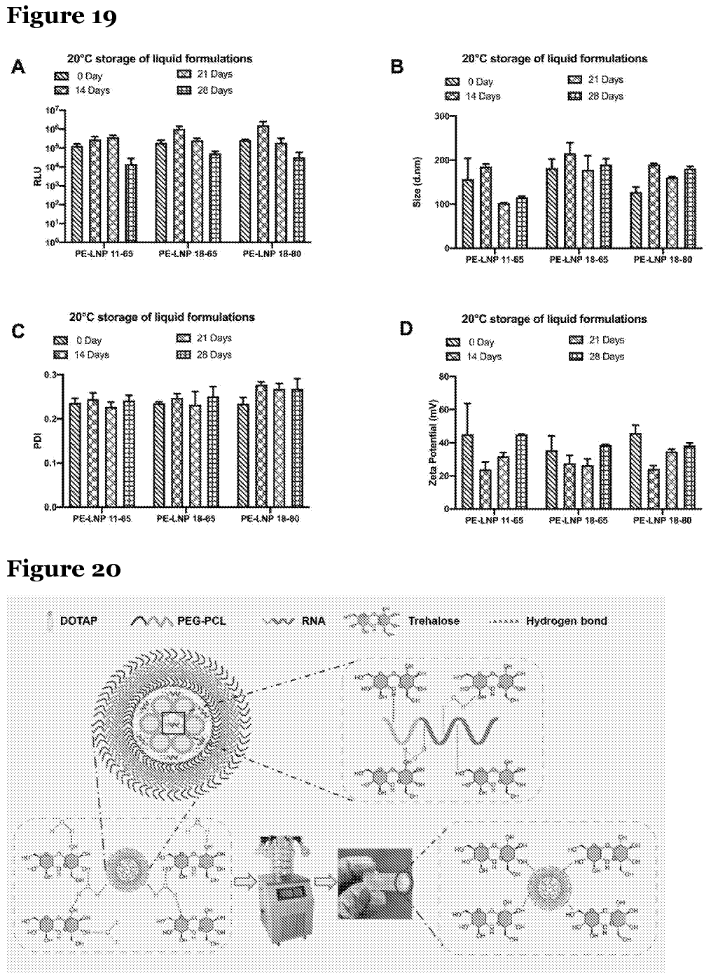

FIG. 19 shows stable storage of 40 μg mL⁻¹ saRNA formulated in PE-LNP 11-65, 18-65 and 18-80, respectively, in aqueous solution at room temperature. The changes in (A) transfection efficiency, (B) DLS size, (C) PDI and (D) zeta potential were recorded after storage in aqueous solution at room temperature for 28 days. The saRNA dose was fixed at 1 μg mL⁻¹ for transfection efficiency;

FIG. 20 shows schematic illustrations of the structure and the preparation process of saRNA- and trehalose-coloaded PE-LNPs;

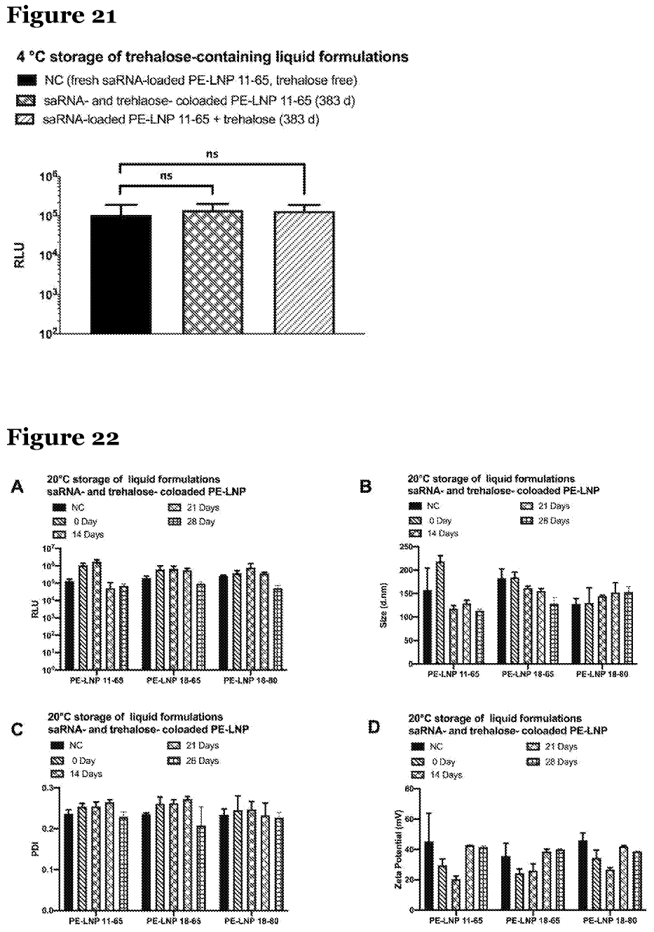

FIG. 21 shows stable storage at 4° C. of 40 μg mL⁻¹ saRNA formulated in PE-LNP 11-65 in aqueous solution containing trehalose: (i) saRNA- and trehalose-coloaded PE-LNP 11-65 (trehalose/saRNA weight ratio=100 for co-encapsulation, followed by mixing with additional trehalose for topping up the total trehalose to 250 mg ml-1); (ii) saRNA-loaded PE-LNP 11-65 mixed with 250 mg mL⁻¹ exterior trehalose. This graph shows the HEK₂₉₃ cell transfection efficiency after storage in aqueous solution at 4° C. for 383 days. The saRNA dose was fixed at 1 μg mL-1;

FIG. 22 shows stable storage of 40 μg mL¹ saRNA coloaded with trehalose in PE-LNP 11-65, PE-LNP 18-65 and PE-LNP 18-80, respectively, in aqueous solution at room temperature (trehalose/saRNA weight ratio=100 for co-encapsulation, followed by mixing with additional trehalose for topping up the total trehalose to 250 mg mL-1). The changes in (A) transfection efficiency, (B) DLS size, (C) PDI and (D) zeta potential were recorded after storage in aqueous solution at room temperature for 28 days. The saRNA dose was fixed at 1 μg mL⁻¹ for transfection efficiency;

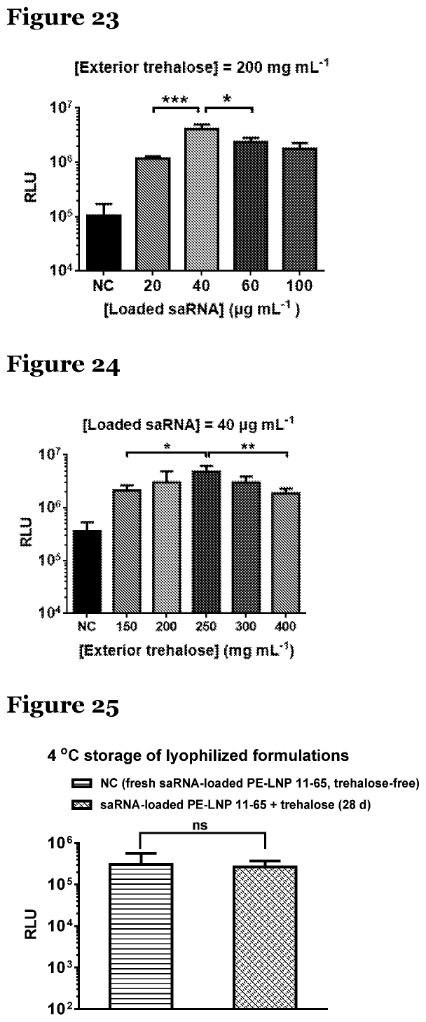

FIG. 23 shows the effect of lyophilization conditions by varying concentrations of saRNA formulated in PE-LNPs in the presence of 200 mg mL⁻¹ exterior trehalose. This graph shows the HEK₂₉₃ cell transfection efficiency of lyophilized PE-LNP 11-65 after immediate rehydration with RNase-free water. The freshly prepared PE-LNP 11-65 (trehalose-free) at the equivalent saRNA dose of 1 μg mL⁻¹ was used as negative control. The single (*) and triple asterisk symbols (***) denote to p<0.05 and p<0.001, respectively;

FIG. 24 shows the effect of lyophilization conditions by varying concentrations of trehalose exterior to PE-LNP 11-65 loaded with 40 μg mL⁻¹ saRNA. This graph shows the HEK₂₉₃ cell transfection efficiency of lyophilized PE-LNP 11-65 after immediate rehydration with RNase-free water. The freshly prepared PE-LNP 11-65 (trehalose-free) at the equivalent saRNA dose of 1 μg mL⁻¹ was used as negative control. The single (*) and double asterisk symbols (**) denote to p<0.05 and p<0.01, respectively;

FIG. 25 shows stable storage at 4° C. after lyophilization of 40 μg mL⁻¹ saRNA formulated in PE-LNP 11-65 in the presence of 250 mg mL⁻¹ exterior trehalose. This graph shows the HEK₂₉₃ cell transfection efficiency of lyophilized PE-LNP 11-65 after storage at 4° C. for 28 days and rehydration with RNase-free water. The freshly prepared PE-LNP 11-65 (trehalose-free) at the equivalent saRNA dose of 1 μg mL⁻¹ was used as negative control. NS represents no significant difference between two groups;

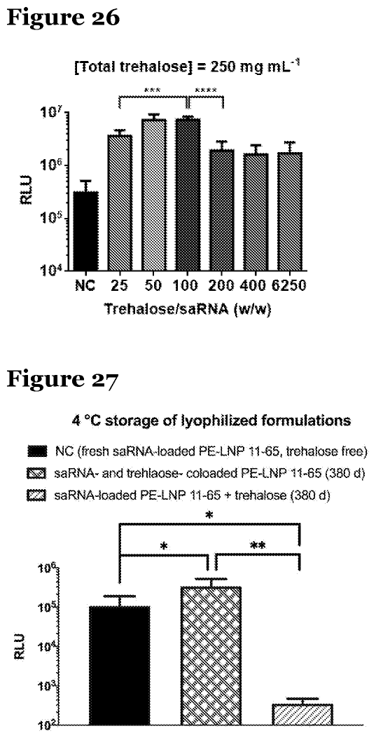

FIG. 26 shows the effect of lyophilization conditions by varying trehalose/saRNA weight ratios. Trehalose was pre-dissolved in the 40 Pg mL-¹ saRNA solution in RNase-free deionized water at various trehalose/saRNA weight ratios for preparing the saRNA- and trehalose-coloaded PE-LNP 11-65. Additional trehalose was then added to the exterior of the obtained nanoparticles for topping up the total trehalose (both interior and exterior of the nanoparticles) to 250 mg mL-1. This graph shows the HEK₂₉₃ cell transfection efficiency of lyophilized saRNA- and trehalose-coloaded PE-LNP 11-65 after immediate rehydration with RNase-free water. The freshly prepared PE-LNP 11-65 (trehalose-free) at the equivalent saRNA dose of 1 μg mL⁻¹ was used as negative control. The triple (***) and quadruple asterisk symbols (****) denote to p p<0.001 and p<0.0001, respectively;

FIG. 27 shows stable storage at 4° C. after lyophilization of 40 μg mL¹ saRNA formulated in PE-LNP 11-65 in the presence of trehalose. This graph shows the HEK₂₉₃ cell transfection efficiency of two different PE-LNP 11-65 samples after lyophilization, storage at 4° C. for 380 days and then rehydration: (i) saRNA- and trehalose-coloaded PE-LNP 11-65 (trehalose/saRNA weight ratio=100 for co-encapsulation, followed by mixing with additional trehalose for topping up the total trehalose to 250 mg mL¹); (ii) saRNA-loaded PE-LNP 11-65 mixed with 250 mg mL⁻¹ exterior trehalose. The saRNA dose was fixed at 1 μg mL-1;

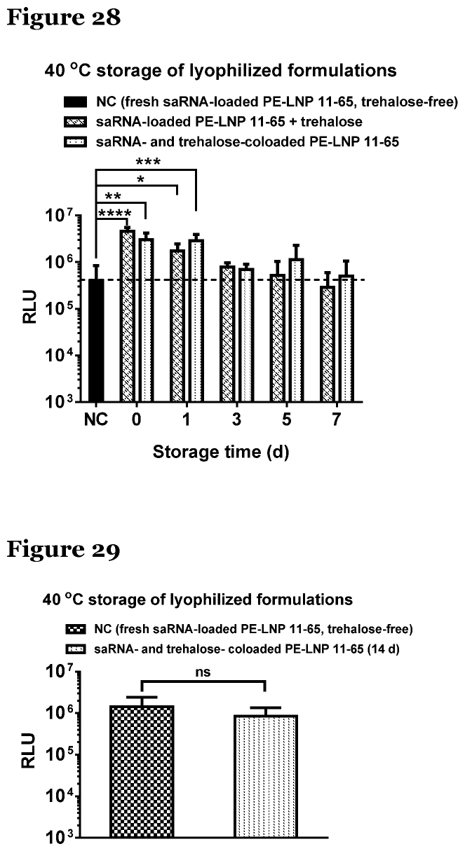

FIG. 28 shows stable storage at 40° C. after lyophilization of 40 μg mL¹ saRNA formulated in trehalose-containing PE-LNP 11-65. This graph shows the HEK₂₉₃ cell transfection efficiency of three different PE-LNP 11-65 samples: (i) saRNA- and trehalose-coloaded PE-LNP 11-65 (trehalose/saRNA weight ratio=100 for co-encapsulation, followed by mixing with additional trehalose for topping up the total trehalose to 250 mg mL-¹) after lyophilization, storage at 40° C. for 0, 1, 3, 5 or 7 days, and rehydration without removing the exterior trehalose; (ii) saRNA-loaded PE-LNP 11-65 mixed with 250 mg mL exterior trehalose after lyophilization, storage at 40° C. for for 0, 1, 3, 5 or 7 days, and rehydration without removing the exterior trehalose; (iii) freshly prepared saRNA-loaded PE-LNP 11-65 (trehalose-free) as negative control. The saRNA dose was fixed at 1 μg mL⁻¹. The single (*), double (*), triple (***) and quadruple asterisk symbols (****) denote to p<0.05, p<0.01, p<0.001 and p<0.0001, respectively;

FIG. 29 shows stable storage at 40° C. after lyophilization of 40 μg mL¹ saRNA formulated in PE-LNP 11-65 in the presence of both interior and exterior trehalose. This graph shows the HEK₂₉₃ cell transfection efficiency of saRNA- and trehalose-coloaded PE-LNP 11-65 (trehalose/saRNA weight ratio=100 for co-encapsulation, followed by mixing with additional trehalose for topping up the total trehalose to 250 mg mL-1) after lyophilization, storage at 40° C. for 14 days and rehydration without removing the exterior trehalose. The freshly prepared saRNA-loaded PE-LNP 11-65 (trehalose-free) at the equivalent saRNA dose of 1 μg mL-¹ was used as negative control. NS represents no significant difference between two groups;

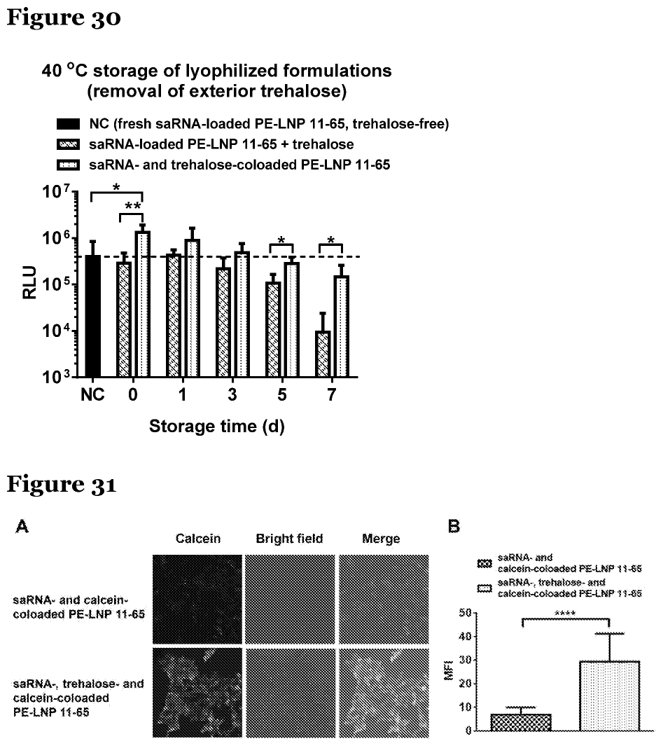

FIG. 30 shows stable storage at 40° C. after lyophilization of 40 μg mL-¹ saRNA formulated in trehalose-containing PE-LNP 11-65. This graph shows the HEK₂₉₃ cell transfection efficiency of three different PE-LNP 11-65 samples: (i) saRNA- and trehalose-coloaded PE-LNP 11-65 (trehalose/saRNAweight ratio=100 for co-encapsulation, followed by mixing with additional trehalose for topping up the total trehalose to 250 mg mL⁻¹) after lyophilization, storage at 40° C. for 0, 1, 3, 5 or 7 days, rehydration and then removal of the exterior trehalose by ultrafiltration centrifugation; (ii) saRNA-loaded PE-LNP 11-65 mixed with 250 mg mL⁻¹ exterior trehalose after lyophilization, storage at 40° C. for 0, 1, 3, 5 or 7 days, rehydration and then removal of the exterior trehalose by ultrafiltration centrifugation; (iii) freshly prepared saRNA-loaded PE-LNP 11-65 (trehalose-free) as negative control. The saRNA dose was fixed at 1 μg mL-1. The single (*) and double asterisk symbols (**) denote to p<0.05 and p<0.01, respectively;

FIG. 31 shows (A) confocal microscopy images of HEK₂₉₃ cells at 4 h after 2 h of treatment with the freshly prepared saRNA- and calcein-coloaded PE-LNP 11-65 or saRNA-, trehalose- and calcein-coloaded PE-LNP 11-65, and (B) MFI of calcein in the confocal microscopy images as analyzed by ImageJ. The quadruple asterisk symbol denotes to p<0.0001;

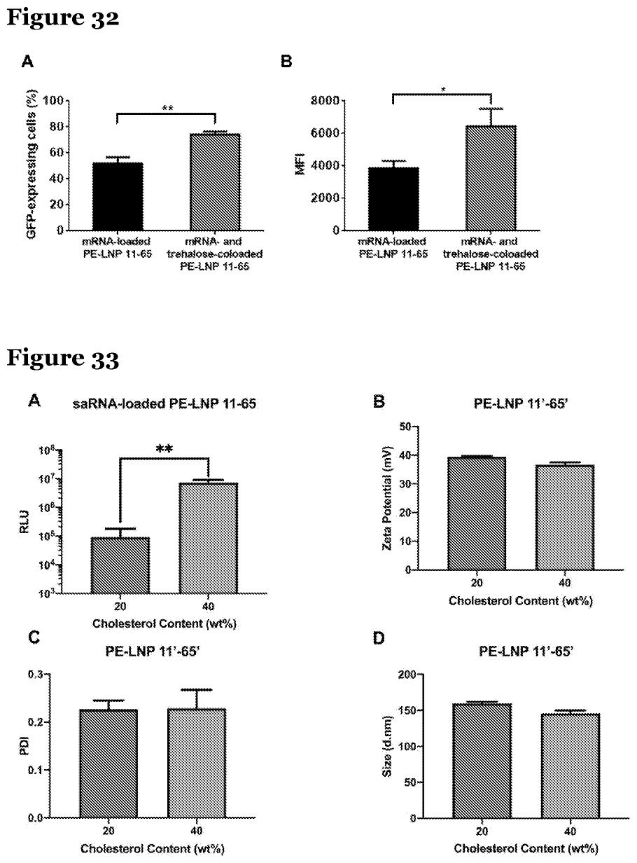

FIG. 32 shows graphs of (A) the percentage and MFI of GFP-expressing HEK₂₉₃ cells treated with the freshly prepared mRNA- and trehalose-coloaded PE-LNP 11-65 (trehalose/mRNA weight ratio=100 for co-encapsulation, followed by mixing with additional trehalose for topping up to the total trehalose at 10 mg mL-1) and saRNA-loaded, trehalose-free PE-LNP 11-65, respectively, as measured by flow cytometry. The single (*) and double asterisk symbols (**) denote to p<0.05 and p<0.01, respectively; and FIG. 33 shows (A) the transfection efficiency of saRNA-loaded PE-LNP 11-65 formulated with cholesterol; (B) zeta potential, (C) PDI and (D) DLS size of PE-LNP 11′-65′ formulated with cholesterol.

### Example 1—Synthesis of PEG-PCL Copolymers with Different Molecular Weights

PEG-PCL copolymers with different molecular weights were synthesized by ring-opening polymerization of ε-caprolactone (ε-CL), which was initiated by mPEG-OH using stannous octoate (Sn(Oct)₂) as catalyst. Briefly, 200 mg mPEG₅ₖ-OH, 470 mg 8-CL (or 200 mg mPEG₂ₖ-OH, 530 mg ε-CL) and 50 mg Sn(Oct)₂ were dissolved in 5 mL anhydrous toluene, and the reaction system was heated to 110° C. under dry nitrogen atmosphere for 48 h²². After the reaction, the mixture was degassed and cooled to room temperature. Then the resulting product was precipitated using excess cold diethyl ether. The polymer was filtered and vacuum dried to constant weight at room temperature.

Characterization of PEG-PCL copolymers ¹H-NMR spectra (in CDCl₃) were recorded on a Jeol 400 MHz NMR spectrometer to characterize the chemical compositions and the degree of polymerization of the PCL block. Gel permeation chromatography (GPC) was employed using the Agilent 1260 Infinity II to determine the polymer molecular weights and molecular weight distributions.

### Results and Discussion

FIG. 1 is the schematic illustration of the preparation procedure of polymer-enveloped lipid nanoparticles (PE-LNPs) and the PE-LNP mediated intracellular delivery of RNA including mRNA and saRNA. PEG-PCL is a biodegradable and biocompatible polymer and both PEG and PCL are approved by FDA for applications in humans. As reported in literature, the molecular weight of amphiphilic polymer plays key role in the self-assembled structure. When the hydrophilic block accounts for 25˜40% of its molecular weight, the amphiphilic copolymer could self-assemble into a robust vesicular structure²³⁻²⁵. The composition and molecular weight of the amphiphilic copolymer can be varied, which allows formation of vesicles with different surface properties, membrane thicknesses and stabilities. PEGs with an M. of 2,000 and 5,000 Da are among the most widely used hydrophilic blocks in drug delivery systems, and the commercially available PEGs (Mₙ=2,000 and 5,000 Da) were thus chosen as initiators in the examples described in this invention. PEG-PCL polymers with two different molecular weights were successfully synthesized by ring-opening polymerization of ε-CL using stannous octoate (Sn(Oct)₂) as catalyst. ¹H-NMR spectra of the products are depicted in FIG. 2A. The number-average molecular weights (M) of the PEG-PCL copolymers, calculated from the integrals of the methylene peak of the caprolactone unit (—COCH₂CH₂CH₂CH₂CH₂O—) at 4.06 ppm and the ethylene peak of the ethylene glycol unit (—CH₂CH₂O—) at 3.60 ppm in the 1H-NMR spectra, were 7,873, 14,473 and 16,580 Da, respectively, which were in good agreement with their theoretical molecular weights (Table 1). This indicates that PEG₂ₖ-PCL₅ₖ, PEG₂ₖ-PCL₃₋₅ₖ and PEG₅ₖ-PCL₁₀ₖ copolymers with desired molecular weights were synthesized successfully. Table 1 also shows the molecular weights of PEG₂ₖ-PCL₅ₖ, PEG₂ₖ-PCL₃₋₅ₖ and PEG₅ₖ-PCL₁₀ₖ copolymers and their relatively low polydispersities (Ð=M_{w}/Mₙ) determined by GPC.

TABLE 1
Theoretical molecular weights (M_{n, th}) and molecular weights
measured by ¹H-NMR (M_{n, NMR}) and GPC (M_{n, GPC}, M_{w, GPC},
polydispersity Ð = M_{w, GPC}/M_{n, GPC}) of PEG₂ₖ-PCL₅ₖ, PEG₅ₖ-PCL_{8.5k}
and PEG₅ₖ-PCL₁₀ₖ

| | M_{n, th} | M_{n, NMR} | M_{n, GPC} | M_{w, GPC} | |
|---|---|---|---|---|---|
| Copolymer | (Da) | (Da) | (Da) | (Da) | Ð |
| PEG₂ₖ-PCL₅ₖ | 7,000 | 7,873 | 5,677 | 9,059 | 1.59 |
| PEG₅ₖ-PCL_{8.5k} | 13,500 | 14,473 | 14,533 | 18,785 | 1.29 |
| PEG₅ₖ-PCL₁₀ₖ | 15,000 | 16,580 | 7,038 | 10,680 | 1.52 |

### Example 2—Preparation of saRNA-Loaded PE-LNPS

PE-LNPs were prepared with a simple one-pot method. Typically, 650 μg PEG₅ₖ-PCL₁₀ₖ and 250 μg DOTAP were co-dissolved in 0.5 mL THF, while 10 μg saRNA was dissolved in 1 mL RNase-free water. The organic and aqueous solutions were quickly mixed and the mixture was kept stirring for 1-2 min at room temperature. The saRNA-loaded PE-LNP 11-65 (N/P=11, polymer/saRNA (w/w)=65) were then obtained after removing THF by rotary evaporation. Various PE-LNPs with different N/P molar ratios or polymer/saRNA weight ratios were prepared similarly. All tips and glassware should be treated prior to use to ensure RNase-free conditions.

### Characterization of PE-LNPs

Size distribution (Z-average) and zeta potential (based on the Smulochowski model) of different PE-LNPs were determined at 25° C. using the Zetasizer μV instrument (Malvern, UK) and ZETA PALS, respectively. The thermal stability of PE-LNPs was evaluated by measuring their size and polydispersity index (PDI) upon temperature ramp from 25° C. to 85° C. through high-throughput dynamic light scattering (HT-DLS) using a DynaPro Plate Reader III (Wyatt, UK).

SEM was used to visualize the morphology of PE-LNPs. One drop of PE-LNP suspension was placed on a graphite surface. The sample was coated with gold using an Ion Sputter after drying. Afterwards, samples were viewed using a JSM-6400 scanning electron microscope (JEOL Ltd, Tokyo, Japan) at an accelerating voltage of 20 kV.

PE-LNP samples for Cryo-TEM (Tecnai F20 G2 at 200 kV) were prepared on a lacey carbon-coated copper grid (Structure Probe Incorporation, PA) using a semi-automated Vitrobot system (Vitrobot Mark II, FEI). Briefly, 4 μL of 1 mg mL⁻¹ PE-LNP solution was casted on top of a carbon grid. The grid was then transferred to a Vitrobot chamber that was at 100% humidity and at 20° C. Rapid immersion of the grid into liquid ethane after 1 s blotting effectively vitrified the sample. The sample was kept under −170° C. using a Gatan 626 cryo holder until successfully transferred into the Cryo-TEM instrument in order to prevent crystalline ice formation. The images were obtained at a defocus of ˜4,000 nm.

The saRNA encapsulation efficiency of PE-LNPs was determined by RiboGreen Assay (Quant-iT™ RiboGreen™ RNA Assay Kit, Thermo Fisher). TE buffer and aqueous RiboGreen working solution were prepared with and without 0.5% Triton X-100 following the manufacturer's instructions. The calibration curve of fluorescence intensity versus saRNA concentration was established. The free (unloaded) saRNA concentration (C_{unloaded}) was determined as follows: samples were diluted with Triton X-100 free TE buffer to an appropriate concentration, mixed with Triton X-100 free RiboGreen working solution and incubated for 15 minutes in the dark. The samples were then pipetted to a black 96-well plate with each well containing 200 μL final mixture. Fluorescence intensity measurements were then taken by a spectrofluorometer (GloMax@ Discover Microplate Reader, Promega, USA) at the excitation and emission wavelengths of 480 and 520 nm, respectively, with three replicates. The total saRNA concentration (Cota) was measured following a similar procedure with TE buffer and RiboGreen working solution containing 0.5% Triton X-100 to lyse the nanoparticles. The encapsulation efficiency of the system could be calculated according to the following equation:

$$Encapsulation efficiency ( \% ) = ( 1 - \frac{{C}_{unloaded}}{{C}_{total}} ) \times 100 \%$$

### Results and Discussion

Various saRNA-loaded PE-LNPs with different N/P molar ratios and polymer/saRNA (w/w) weight ratios were obtained. The PE-LNP with N/P=5 and polymer/saRNA (w/w)=65 is named as PE-LNP 5-65. The other PE-LNPs are assigned according to the same rule. The size distribution, PDI and zeta potential of PE-LNPs with different compositions have been summarized in FIG. 2C. saRNA (˜9,500 nt) is required to be encapsulated into a carrier which can provide the protection and facilitate the intracellular delivery of saRNA. FIG. 2B shows that the saRNA-loaded PE-LNPs were monodisperse. As listed in FIG. 2C, the PE-LNPs assembled from PEG₅ₖ-PCL₁₀ₖ showed the average hydrodynamic size of 113.1 f 0.3-134.2 f 0.6 nm in diameter and the low PDI of 0.21 f 0.03 0 0.35±0.02. The PE-LNPs composed of PEG₂ₖ-PCL₅ₖ showed the larger size (133.9±0.5˜ 149.6±0.4 nm) and higher PDI (0.49 f 0.09-0.59±0.10). It indicates that the PE-LNPs self-assembled from PEG₅ₖ-PCL₁₀ₖ exhibited the more stable structure, which could be attributed to the longer hydrophilic PEG chain length and the thicker polymer capsule layer.

The N/P molar ratio is a critical factor controlling the saRNA loading capacity and endosomal escape efficiency. With increasing the N/P ratio from 5 to 18, the zeta potential of the PE-LNPs composed of PEG₅ₖ-PCL₁₀ₖ (polymer/saRNA weight ratio=65) increased significantly from +29.0 f 0.9 mV to +44.5 f 0.8 mV due to the increased amount of the cationic lipid. However, when the polymer/saRNA weight ratio in the PE-LNPs increased from 65 to 80, zeta potential of the PE-LNPs composed of PEG₅ₖ-PCL₁₀ₖ (N/P=18) decreased from +44.5±0.8 mV to +40.2 f 0.9 mV, which could be due to the enhanced shielding effect of the polymer capsule layer. The PE-LNPs composed of PEG₂ₖ-PCL₅ₖ showed the same trends.

PEG₅ₖ-PCL₁₀ₖ and DOTAP were used to prepare the RNA-loaded PE-LNP m-n for further investigation in the exemplified work unless specified, with the numbers m and n denoting to the N/P molar ratio and polymer/RNA weight ratio, respectively. FIG. 3 shows the changes in encapsulation efficiency of PE-LNP with various N/P molar ratios (polymer/saRNA weight ratio=65). When the N/P molar ratio increased from 5 to 11, a significant increase by 20% in the encapsulation efficiency was observed. The encapsulation efficiency of PE-LNP 11-65 was 93.6 f 3.3%, indicative of a very high RNA loading capacity of the nano-formulations.

The counterpart of PE-LNP m-n without the payload is named as PE-LNP m‘-n’. The structure of PE-LNPs without RNA loading was investigated by Cryo-TEM. The Cryo-TEM micrographs in FIG. 4 show that PE-LNPs displayed a clear outer layer. The amphiphilic PEG-PCL polymer could self-assemble into a polymersome structure which contains a hydrophilic core. The darker outer layer should be attributed to the hydrophobic PCL polymer with higher contrast. In the core there were a number of black dots which were thought to be the aggregated DOTAP lipid nanoparticles. DOTAP is amphiphilic where, the polar part likes water, also referred to as hydrophilic, and the non-polar part as the hydrophobic part tries to stay away from water. As the inner PEG-PCL vesicular structure is hydrophilic, the ‘hydrophobic effect’ ²⁶ (which acts to minimize contact between lipid hydrocarbon tails and the aqueous environment) of the DOTAP tails together with others including van der Waals interactions and head group hydrogen bonding determines the self-assembled behaviour of DOTAP. Therefore, the black dots, which showed the darker contrast due to the poor electron penetrability, likely consisted of unsaturated hydrophobic hydrocarbon tails present in DOTAP.

Accordingly, DOTAP self-assembled into “sponge-like” aggregates, which were encapsulated into the aqueous core of the polymersome and divided the internal space into sub-compartments though hydrophobic interactions of the hydrocarbon tails. Compared with liposomes and polymersomes, the internal structure of PE-LNPs provides a significantly higher surface area which is favorable for nucleic acid loading. Further, the PEG-PCL shell can not only prevent the degradation of RNA but also increase the stability of the PE-LNP system due to its higher mechanical strength than the lipid bilayer.

The inventors evaluated the thermal stability of PE-LNP 11′-65′ 11′-80′, 18′-65′ and 18′-80′. As shown in FIG. 5, the DLS size and the PDI of the PE-LNPs were irresponsive to temperature change within the range of 25° C. to 85° C., demonstrating good structural and colloidal stability upon temperature change.

### Example 3—Investigation of the PE-LNP Structure Through Fluorescence Resonance Energy Transfer (Fret) Analysis

FRET experiments were carried out to further investigate the PE-LNP structure using a fluorometer (FluoroMax-4, Horiba Scientific). For the donor/acceptor pair of N-(7-nitro-2-1,3-benzoxadiazol-4-yl)-1,2-dipalmitoyl-sn-glycero-3-phosphoethanolamine (NBD-PE)/N-(lissamine rhodamine B sulfonyl)-1,2-dipalmitoyl-sn-glycero-3-phosphoethanolamine (Rhod-PE), the excitation wavelength was set at 460 nm and emission spectra were collected from 480 to 630 nm.

PE-LNPs were incorporated with the donor NBD-PE (4 mM) and the acceptor Rhod-PE (4 mM). Briefly, for various PE-LNPs such as PE-LNP 5′-65′, PE-LNP 11′-65′ and PE-LNP 18′-65′ without saRNA loading, NBD-PE and Rhod-PE were co-dissolved in 0.5 mL THF (containing pre-dissolved PEG-PCL and DOTAP) at the desired ratio, followed by quick mixing with 1 mL H₂O. The mixture was stirred for 1-2 min at room temperature and THF was then removed by rotary evaporation (see the PE-LNPs preparation section in Example 2 above). Control groups without polymer (i.e., DOTAP liposomes) were prepared for comparison. The molar concentrations of DOTAP in the control liposomes were either the same as the molar concentration of DOTAP (or the total molar concentration of both PEG-PCL and DOTAP) in the corresponding PE-LNPs. For example, the molar concentrations of DOTAP and PEG₅ₖ-PCL₁₀ₖ in PE-LNP 5′-65′ were 143 mM and 43 mM, respectively. Therefore, the corresponding control liposomes were denoted as DOTAP-143 and DOTAP-186, respectively. In addition, the respective donor-labelled (NBD-PE only) PE-LNPs were also prepared to calculate the FRET efficiency E₁ according to the following equation:

$${E}_{1} ( \% ) = \frac{{I}_{D} - {I}_{DA}}{{I}_{D}} \times 100 \%$$

where I_{D} and I_{DA} are the donor fluorescence intensities at 530 nm of PE-LNPs labelled with the donor NBD-PE only and PE-LNPs co-labelled with both the donor NBD-PE and the acceptor NBD-PE, respectively.

In another experiment, PE-LNP 11′-65′ were labelled with NBD-PE (4 mM) and Rhod-PE (4 mM) separately. Then, PE-LNP 11′-65′ (NBD-PE only) were mixed with PE-LNP 11′-65′ (Rhod-PE only) in equal volumes under shaking at 100 rpm for 5 min, 1 h and 2 h, respectively. The control DOTAP liposomes were also labelled with NBD-PE (4 mM) and Rhod-PE (4 mM) separately, and the molar concentration of DOTAP in the control liposomes was equivalent to that of DOTAP in the corresponding PE-LNPs. The blank PE-LNPs and DOTAP liposomes without labelling were prepared for comparison. The FRET efficiency E₂ at the specified time point was calculated according to the following equation:

$${E}_{2} ( \% ) = \frac{{I}_{D} - {I}_{DM}}{{I}_{D}} \times 100 \%$$

where I_{D} is the donor fluorescence intensity at 530 nm of PE-LNPs labelled with NBD-PE after mixing with blank samples, and I_{DM} is the donor fluorescence intensity of PE-LNPs labelled with NBD-PE after mixing with PE-LNPs labelled with Rhod-PE.

### Results and Discussion

To further verify that lipid nanoparticles are formed in the core of nanostructures, FRET analysis using the NBD-PE and Rhod-PE pair was applied to investigate the spatial arrangement of PEG-PCL and DOTAP. The efficiency of energy transfer between the donor fluorophore NBD (4.0 mM) and the acceptor fluorophore Rhod (4.0 mM) is dependent on their distance on the membrane layer. The most common structure formed by mixing an amphiphilic polymer with a lipid is a hybrid vesicular architecture with mixed membrane composition²⁷⁻²⁹. As shown in FIG. 6B, excitation of the donor NBD at 460 nm resulted in the fluorescent spectrum with an enhanced emission intensity of the acceptor Rhod in PE-LNP 5′-65′, showing a FRET efficiency of 93.7±0.2%. Assuming that PEG-PCL and DOTAP were self-assembled into a homogeneous hybrid membrane, the FRET efficiency of the control DOTAP-186 liposomes (assembled from DOTAP at the same molar concentration with combined polymer and lipid molecules present in PE-LNP 5′-65′) should be at a similar level to that of PE-LNP 5′-65′ due to the similar distance between the donor NBD and the acceptor Rhod (FIG. 6A). In fact, the FRET efficiency of PE-LNP 5′-65′ was significantly higher than that of DOTAP-186 liposomes (85.9±0.3%). Similarly, assuming that PEG-PCL and DOTAP were self-assembled into a homogeneous hybrid membrane, the FRET efficiency of the control DOTAP-143 liposomes (assembled from DOTAP at the same molar concentration with the DOTAP present in PE-LNP 5′-65′) should be higher than that of PE-LNP 5′-65′ due to the shorter distance between the donor NBD and the acceptor Rhod. However, although being self-assembled from fewer molecules DOTAP-143 liposomes displayed a similar level of FRET efficiency (92.9 f 0.3%) with PE-LNP 5′-65′. Similar trends were observed for PE-LNP 11′-65′ and PE-LNP 18′-65′ (FIGS. 6C-6D). The significant difference in the FRET efficiency suggests that there was no existence of homogeneous polymer-lipid hybrid membrane in PE-LNPs.

Further work was then carried out to validate the formation of lipid nanoparticles in the core of PE-LNPs. PE-LNP 11′-65′ were labeled with the donor NBD-PE (4.0 mM) or the receptor Rhod-PE (4.0 mM) separately. Then, PE-LNP 11′-65′ (NBD) were mixed with PE-LNP 11′-65′ (Rhod) in an equal volume under shaking at 100 rpm for up to 2 h. The control DOTAP-358 liposomes, where the number (i.e., 358) denotes to an equivalent molar concentration of DOTAP present in PE-LNP 11′-65′, were also labeled with the donor NBD-PE (4.0 mM) or receptor the receptor Rhod-PE (4.0 mM) separately for comparison. After DOTAP-358 liposomes (NBD) were mixed with DOTAP-358 liposomes (Rhod) for incubation under the same condition, an immediate increase in the FRET efficiency to 17.1% was observed, followed by a gradual increase to 22.0% after 1 h (FIGS. 7B-7D). This was due to the lipid mixing between the DOTAP liposomes, which led to a decrease in the distance between the donor NBD and the acceptor Rhod. However, no obvious FRET efficiency was observed after mixing of PE-LNP 11′-65′ (NBD) with PE-LNP 11′-65′ (Rhod) for 1 h, followed by only a slight increase to 3.4% after extended incubation for 2 h. The negligible FRET efficiency suggests the formation of lipid nanoparticles in the core of PE-LNPs (FIG. 7A).

### Example 4—In Vitro HEK₂₉₃ Cell Transfection by saRNA-Containing PE-LNPs

Transfection efficiency of saRNA-loaded PE-LNPs (saRNA in the nanoparticle core) For the firefly luciferase (fLuc) assay, HEK₂₉₃ cells were seeded in a 96-well plate at a density of 5×10⁴/well and cultured in the DMEM medium supplemented with 10% (v/v) FBS and 1% (v/v) penicillin/streptomycin for 48 h to reach 60˜80% confluence before transfection. Following the removal of spent medium, 100 μL of fresh serum-free or complete DMEM medium was added to each well, which was then added in replicates of 5 with 10 μL of fLuc saRNA-loaded PE-LNPs at different N/P molar ratios and polymer/saRNA (w/w) weight ratios (equivalent to 1 μg mL-¹ saRNA). After 4 h of incubation, the transfection medium was replaced with the fresh complete DMEM medium. The fLuc activity, expressed as relative light units (RLU), in 50 μL of medium from the transfected cells following 24 h of treatment with 50 μL of fLuc substrate was assayed using a GloMax® Microplate Reader (Promega).

Transfection efficiency of saRNA-attached PE-LNPs (saRNA on the nanoparticle surface) fLuc saRNA-attached PE-LNPs were prepared by method 2 (M₂) for comparison.

Briefly, 650 μg of PEG₅ₖ-PCL₁₀ₖand 400 μg of DOTAP were co-dissolved in 0.5 mL of THF. The resulting organic solution was quickly added to 1 mL of Rnase-free water and stirred for 1-2 min at room temperature. THF was then removed by rotary evaporation to obtain the blank PE-LNPs. After that, 10 μg of fLuc saRNA was added in the blank PE-LNP solution. After vortexing for 30 s, fLuc saRNA-attached PE-LNPs were obtained. The transfection efficiency of fLuc saRNA-attached PE-LNPs in the absence or in the presence of FBS was determined using the abovementioned method in Example 4. The PEI/fLuc saRNA complexes and fLuc saRNA-loaded DOTAP lipid nanostructures were prepared as controls.

Transfection Efficiency of saRNA-Loaded PE-LNPs and saRNA-Attached PE-LNPs after Storage at 4° C.

100 μL of Rnase-free PBS (10×) buffer (or Rnase-free water) was added to 900 μL of fLuc saRNA-loaded PE-LNP or fLuc saRNA-attached PE-LNP solution, and the resulting samples were storage at 4° C. HEK₂₉₃ cells were then transfected with these samples which were freshly prepared or stored at 4° C. for 5 days and the transfection efficiency was measured following the abovementioned method in Example 4.

### Results and Discussion

The effects of PEG-PCL molecular weights, N/P molar ratios and polymer/saRNA(w/w) weight ratios on the transfection efficiencies of various PE-LNPs were evaluated in HEK₂₉₃ cells. FIGS. 8A and 8B display the transfection efficiencies of PE-LNPs with the fixed polymer/saRNA (w/w) weight ratio of 65 but different N/P molar ratios. For the PE-LNPs composed of PEG₂ₖ-PCL₅ₖ, the transfection efficiency was improved by nearly four orders of magnitude when the N/P molar ratio increased from 5 to 16, and then decreased with further increasing the N/P molar ratio. As comparison, the PE-LNPs composed of PEG₅ₖ-PCL₁₀ₖ showed a similar trend of transfection efficiency, but with the higher transfection efficiency at a lower N/P molar ratio of 5 and the higher peak transfection efficiency at the N/P molar ratio of 18.

FIGS. 8C and 8D depict the transfection efficiencies of the PE-LNPs with the fixed N/P molar ratio but varying polymer/saRNA weight ratios. For the PE-LNPs composed of PEG₂ₖ-PCL₅ₖ, the transfection efficiency increased by two orders of magnitude when the polymer/saRNA weight ratio increased from 40 to 65. The change of transfection efficiency became insignificant with further increasing the polymer/saRNA weight ratio to 90 (FIG. 8C). However, in terms of the PE-LNPs composed of PEG₅ₖ-PCL₁₀ₖ, the transfection efficiency was at a similar level within the polymer/saRNA weight ratio range (40˜90) tested (FIG. 8D). This could be due to the enhanced formation of a thicker polymer shell layer from PEG₅ₖ-PCL₁₀ₖ with a higher molecular weight. The resulting thicker polymer shell layer could offer improved protection of saRNA, which was confirmed by the marginal changes in the transfection efficiency between the PE-LNP samples in the absence (FIG. 8D) and in the presence of FBS (FIG. 8F) at the polymer/saRNA weight ratio from 65 to 90. In contrast, the PE-LNPs composed of PEG₂ₖ-PCL₅ₖ showed considerably reduced transfection efficiencies in the presence of FBS (FIG. 8E). The high serum compatibility, which was attributed to the enhanced stability of PE-LNPs with a thicker polymer shell layer, is favorable because many other cationic nucleic acid delivery systems with high in vitro transfection efficiencies demonstrated low in vivo efficacies due to the adsorption of serum proteins. Therefore, the PE-LNPs composed of PEG₅ₖ-PCL₁₀ₖ were chosen in the further work.

RNA molecules are very unstable and can be readily hydrolyzed/degraded. Two different RNA-loading methods were employed to compare the stability of RNA loaded in the core and attached on the surface of PE-LNPs. As shown in FIG. 9A, the saRNA-loaded PE-LNP 18-80 and saRNA-attached PE-LNP 18-80 showed comparable transfection efficiencies in the absence of FBS, which were one order of magnitude higher than the PEI/saRNA complexes and four to five orders of magnitude higher than the saRNA-loaded DOTAP lipid nanostructures. It is interesting to note that the saRNA-loaded PE-LNP 18-80 was FBS-compatible, while the presence of FBS caused a reduction in the transfection efficiency of saRNA-attached PE-LNP 18-80 by one order of magnitude (FIG. 9B). As comparison, the transfection efficiency of the PEI/saRNA complexes decreased by three orders of magnitude upon incubation with FBS. That means, the saRNA-loaded PE-LNP 18-80 exhibited four orders of magnitude higher transfection efficiency than the PEI/saRNA complexes in the presence of FBS.

The inventors further evaluated the stability of RNA loaded in the core and attached on the surface of PE-LNPs after storage in RNase-free water or PBS at 4° C. As shown in FIGS. 9C and 9D, after storage in the liquid formulation for 5 days, saRNA-loaded PE-LNP 18-80 showed no reduction in the transfection efficiency, whilst the transfection signal of saRNA-attached PE-LNP 18-80, although still relatively high (>10⁶), was half an order of magnitude lower compared to the freshly prepared sample.

These results suggest that the PE-LNP systems show the favorable serum stability and high encapsulation of biological payloads, in particular readily hydrolyzed/degraded RNA molecules, in the nanoparticle core can provide the optimal protection. This is because the hydrophilic PEG corona of PE-LNPs can reduce the protein absorption and the hydrophobic PCL layer can protect RNA from the harsh external environment.

### Example 5—In Vitro Transfection in Interferon-Competent HeLa Cells by saRNA-Loaded PE-LNPs

Transfection efficiency of saRNA-loaded PE-LNPs fLuc encoded saRNA was formulated in the interior of PE-LNP 11-65 by the same method described above in Example 2. HeLa cells were seeded and transfected following the abovementioned method in Example 4, where a titration was performed by changing the saRNA dose to 0.1, 0.5, 1, 3, 5 and 7 μg mL⁻¹, respectively.

### Results and Discussion

In addition to inefficient protein expression, clinical applications of RNA systems are restricted by their high innate immunogenicity. It is crucial to achieve a good balance between protein expression and innate immune response. Toll-like receptors (TLRs) 3, 7, 8 and 9 are intracellular sensors of nucleic acids residing in endoplasmic reticulum, endosomes and lysosomes. Upon detection of foreign nucleic acids, the intracellular TLRs activate various signalling pathways and trigger the production of cytokines that could lead to limited protein expression or even adverse effect on the patient. Since HeLa cells express TLR₃, transfection efficiency on HeLa cells were evaluated to examine the ability of PE-LNPs to prevent recognition by the intracellular TLRs.

FIGS. 10A and 10B depict the cell transfection titre of PE-LNPs on interferon-competent HeLa cells. The four formulations tested, PE-LNP 11-65, PE-LNP 18-65, PE-LNP 11-80 and PE-LNP 18-80, shared the similar trend of transfection efficiency, increasing with the saRNA dose until the optimal dose of 3 μg mL⁻¹ where the maximum transfection efficiency with RLU of up to 107 was reached. All four PE-LNP formulations exhibited a promising level of transfection efficiency, which indicates effective prevention of TLR recognition by the PE-LNP structure. This is attributed to the polymer shell, of which the hydrophilic block provides a hydration layer, preventing recognition by the TLRs as well as direct interactions between the cationic lipid core with the cells, resulting in effective transfection on interferon-competent cells. For lower doses of saRNA (0.1 to 5 μg mL⁻¹ when polymer/saRNA weight ratio=65; 0.5 μg mL-¹ to 3 μg mL-1when polymer/saRNA weight ratio=80), the transfection efficiency increased by approximately one order of magnitude when the N/P molar ratio increased from 11to 18. This could be due to the higher N/P molar ratio leading to more efficient release of the payload in the endosomal compartment. It might thus lower the chances of activating of TLRs that are localised to the endosomes.

These results also show that the PE-LNPs can effectively deliver nucleic acid payloads into various cell types, including human embryonic kidney cells (Example 4), cancer cells (Example 5) and T lymphocyte cells (Example 6).

### Example 6—PE-LNP Mediated Intracellular Delivery of mRNA to Suspension Jurkat Cells

To further demonstrate the ability of PE-LNPs to deliver different payloads to different cell lines, the inventors used various PE-LNPs for intracellular delivery of green fluorescent protein (GFP)-encoding messenger RNA (mRNA) to suspension Jurkat cells. The GFP mRNA-loaded PE-LNPs were prepared by the same method described above in Example 2 for saRNA-loaded PE-LNPs. Briefly, Jurkat cells were gently pipetted to get resuspended and then counted by a Vi-CELL XR Cell Viability Analyzer (Beckman Coulter, USA). Cells were washed twice and then resuspended at the density of 2×10⁶ cells mL¹ in pre-warmed (37° C.) un-supplemented serum-free RPMI-1640 medium. 1 mL of cell suspension was seeded into each well of a 12-well plate. Then, GFP mRNA-loaded PE-LNPs were added dropwise at 2 μg mRNA per well, immediately followed by gentle pipetting to mix the culture thoroughly. After incubation at 37° C. for 4 h, the cells were washed twice and resuspended in 1 mL complete RPMI-1640 medium for further incubation at 37° C. overnight. Some of the sample wells were used for the viability test through cell counting by a Vi-CELL XR Cell Viability Analyzer. The rest sample wells were washed with PBS twice and cells were resuspended in 100 μL PBS containing the LIVE/DEAD™ Fixable Aqua Dead Cell Stain (Thermo Fisher, USA) and incubated for 30 min. After washing with PBS, each sample was observed by fluorescence microscopy (EVOS Floid Imaging System, Thermo Fisher) and quantitatively analysed by flow cytometry (Canto, BD, USA) to examine the GFP expression in viable Jurkat cells.

### Results and Discussion

FIG. 11 shows that GFP expression was negligible in Jurkat cells treated with mRNA-loaded PE-LNP 5-65. When the polymer/mRNA weight ratio was fixed at 65, the percentage of GFP-expressing Jurkat cells increased considerably to over 80% with increasing the N/P molar ratio from 5 to 11, followed by a further enhancement by approximately 10% with further increasing the N/P molar ratio to 18 (FIG. 11B). The mean fluorescence intensity (MFI) in Jurkat cells treated with those mRNA-loaded PE-LNPs exhibited a similar dependence on the N/P molar ratio change from 5 to 11. However, no significant enhancement was observed with further increasing the N/P molar ratio to 18 (FIG. 11C). FIG. 11D depicts that Jurkat cells were well tolerated by the PE-LNPs, with PE-LNP 5-65 and PE-LNP 11-65 showing the high cell viability of more than 80% after 4 h of treatment. Although a slightly increased cytotoxicity was observed with increasing the N/P molar ratio to 18, PE-LNP 18-65 still presented a relatively high cell viability at 70.4±1.8%. The results demonstrate that the inventors' PE-LNPs can also deliver mRNA to suspension cell lines successfully.

### Example 7—Mechanism of Cellular Uptake and Endosomal Escape of saRNA- and FITC-Coloaded PE-LNPs

500 μg of FITC, 800 μg of PEG₅ₖ-PCL₁₀ₖ and 400 μg of DOTAP were co-dissolved in 0.5 mL of THF, 10 μg of saRNA was dissolved in 1 mL of Rnase-free water. The organic and aqueous solutions were quickly mixed and the mixture was kept stirring for 1-2 min at room temperature. THF was removed by rotary evaporation and free FITC was removed by centrifugation in ultrafiltration centrifugal tube (MWCO=3000) at 3000 rpm for 15 min to obtain the saRNA- and FITC- coloaded PE-LNPs.

The cellular uptake mechanism of the nanoscale system was investigated by laser scanning confocal microscopy. A 2 mL amount of HEK₂₉₃ cells (2×10⁵ cells per dish) was seeded into a 35 mm glass-bottom culture dish and cultured for 24 h. After pre-incubation at 4° C. for 1 h, saRNA- and FITC-coloaded PE-LNPs (final saRNA concentration at 2 μg per dish) were added and cells were further incubated at 4° C. for 4 h. HEK₂₉₃ cells were treated with saRNA- and FITC-coloaded PE-LNPs at 37° C. for 4 h as control. After treatment at 4° C. or 37° C., cells were washed with PBS and fixed with a 4% paraformaldehyde solution for 10 min, and the nuclei and lysosomes were stained with Hoechst 33342 (5 μg mL¹) and LysoTracker-Red (50 nM), respectively for 5 min. The cells were then imaged using a Leica SP8 Inverted confocal microscope and the fluorescence colocalization of FITC and LysoTracker-Red was analysed by Image J.

To further investigate the mechanism of cellular uptake and intracellular transport of saRNA- and FITC-coloaded PE-LNPs, HEK₂₉₃ cells were seeded at a density of 5×10⁵/well in a 6-well plate for 24 h. Firstly, cells were pre-incubated with the following inhibitors, respectively for 1 h: chlorpromazine hydrochloride (10 μg mL⁻¹), methyl-β-cyclodextrin (MβCD, 5 mM), filipin (5 μg mL⁻¹), amiloride (1 mM), genistein (40 μg mL-1) and nystatin (40 μg mL⁻¹). Then, the saRNA- and FITC-coloaded PE-LNPs (containing 2 μg saRNA) were added to each well and co-incubated with the inhibitors for another 1 h. Finally, cells were washed with pre-cooled PBS solution for three times and was analyzed by flow cytometry (Fortessa I).

The uptake and intracellular trafficking of saRNA- and FITC-coloaded PE-LNPs were also studied by confocal microscopy. A 2 mL amount of HEK₂₉₃ cells (2×10⁵ cells per dish) was seeded into a 35 mm glass-bottom culture dish and cultured for 24 h. After pre-incubation with MβCD (5 mM) for 1 h, saRNA- and FITC-coloaded PE-LNPs (containing 2 μg saRNA) were added to the culture dish and co-incubated with MβCD for 4 h. Cells were then washed with PBS and fixed with 4% paraformaldehyde solution for 10 min, and the nuclei and lysosomes were stained with Hoechst 33342 (5 μg mL⁻¹) and LysoTracker-Red (50 nM), respectively for 5 min. Finally, the cells were imaged using a Leica SP8 Inverted confocal microscope.

### Results and Discussion

It is crucial to design a nano-carrier which can release endocytosed biological molecules into the cytoplasm by endosomal escape before they are trafficked to lysosomes for degradation³⁰ (FIG. 1B). In this work, FITC was co-loaded with saRNA in PE-LNP 18-80 in order to investigate the endocytosis pathway and intracellular trafficking. After treatment at 37° C. for 4 h, strong diffusion green fluorescence of FITC was visualised by confocal microscopy (FIG. 12A). ImageJ analysis suggests that the signal of fluorescence co-localization between the saRNA- and FITC-coloaded PE-LNPs (green) and endosomes/lysosomes (red) was negligible, indicative of the successful PE-LNP mediated endosomal escape of the payload into cytoplasm (FIG. 12B). As comparison, the intracellular green fluorescence intensity of FITC was considerably reduced to a fairly low level after incubation at 4° C. for 4 h, suggesting that the cellular uptake of PE-LNPs was via the energy-dependent endocytosis pathway (FIGS. 12A and 12B). Furthermore, various endocytic inhibitors were used to further investigate the specific endocytic mechanism of PE-LNPs. FIGS. 12C and 12D show that the intracellular green fluorescence intensity was significantly decreased after treatment with the inhibitor MβCD, suggesting that lipid-raft mediated endocytosis is the main cellular uptake pathway of PE-LNPs.

### Example 8—Hemolysis and Cell Viability Test

The biocompatibility of saRNA-loaded PE-LNPs was investigated using a hemolysis method. Briefly, defibrinated sheep erythrocytes (RBCs) were centrifuged at 1500×g for 10 min at 4° C. and washed with PBS for three times. The cell pellets were resuspended into a 5% (v/v) erythrocyte suspension with PBS. A 100 μL aliquot of different PE-LNPs containing 1 μg saRNA were added into 0.9 mL of the RBC suspension in a centrifuge tube. Treatment of the RBC suspension with deionized water was used as the positive control. After incubation at 37° C. for 1 h, the RBC suspension was centrifuged and 100 μL of the supernatant was transferred to a 96-well plate, and the absorbance (A) was measured at 540 nm using a spectrofluorometer (GloMax® Discover Microplate Reader, Promega, USA). The relative hemolysis was calculated according to the following equation:

$$Hemolysis ( \% ) = \frac{{A}_{sample} - {A}_{buffer}}{{A}_{deionized water} - {A}_{buffer}} \times 100 \%$$

The cytotoxicity of saRNA-loaded PE-LNPs against HEK₂₉₃ cells was measured using alamarBlue assay. HEK₂₉₃ cells were seeded in a 96-well plate at a density of 5×10⁴ cells/well. After incubation for 24 h, the cells were treated with various PE-LNPs loaded with 1 μg mL-¹ saRNA for 4 h. Then, 10 μL of alamarBlue HS reagent (5 mg mL-1) was added to each well. According to the manufacturer's instructions, after further incubation for 4 h, the absorbance of each well at 570 nm was measured using a Spectrofluorometer (GloMax@ Discover Microplate Reader, Promega, USA). The cytotoxic effect was determined from the absorbance readings.

### Results and Discussion

The hemolytic activity and non-specific cell cytotoxicity are typical issues associated with cationic carriers due to their strong interaction with negatively changed cell membranes. It is interesting to note that the hemolysis rates of various PE-LNPs were all below 10% after 1 h of treatment (FIG. 13A), suggesting the high biocompatibility. FIG. 13B shows that HEK₂₉₃ cells were well tolerated by the saRNA-loaded PE-LNPs. At the fixed saRNA dose of 1 μg mL-¹, PE-LNP 5-65 and PE-LNP 11-65 displayed the very high cell viability of 92.3 f 1.8% and 86.7 f 1.4%, respectively after 24 h of treatment. Although an increased cytotoxicity was observed with increasing the N/P molar ratio to 18, PE-LNP 18-65 and PE-LNP 18-80 still showed the relatively high cell viability at 73.0 f 4.9% and 73.6 f 7.8%, respectively. These results demonstrate that PE-LNPs had low cytotoxicity and favorable biocompatibility, which could be due to the protection provided by the hydrophilic PEG corona and hydrophobic PCL layer surrounding the biological payload in the core of PE-LNPs.

### Example 9—In Vivo Bioluminescence Imaging and Immunogenicity

### In Vivo Bioluminescence Imaging

Female BALB/c mice (Charles River, UK), 6-8 weeks of age, were placed into groups of n=5 and housed in a fully acclimatized room with free access to food and water. All animals were handled in accordance with the UK Home Office Animals Scientific Procedures Act of 1986 in accordance with an internal ethics board and a UK government-approved project and personal license. Animals were conceded an adaption time of at least 7 days before the beginning of the experiments. Mice were injected intramuscularly (IM) in both hind leg quadriceps muscles with 5 μg of fLuc saRNA formulated in the interior of DOTAP lipid nanostructures (negative control), PE-LNP 5-65, PE-LNP 11-65, PE-LNP 18-65 and PE-LNP 18-80, respectively or formulated on the surface of PE-LNP 18-80 M₂ (prepared with Method 2). After 7 days, the mice were injected intraperitoneally with 100 μL of XenoLight RediJect D-Luciferin Substrate (Perkin Elmer, UK) and allowed to rest for 10 min. Mice were then anesthetized using isoflurane and imaged on an In Vivo Imaging System FX Pro (Kodak Co., Rochester, NY, USA) equipped with Molecular Imaging Software Version 5.0 (Carestream Health, USA) for 10 min. A signal from each injection site was quantified using an equal detection area, using Molecular Imaging Software, and expressed as RLU.

In Vivo Immunogenicity of HA saRNA-Loaded PE-LNPs

Female BALB/c mice (Charles River, UK), 6-8 weeks of age, were placed into groups of n=5. Mice were immunized IM in one hind leg quadriceps muscle with 1 μg of saRNA encoding H₁ hemagglutinin of the Cal/09 virus (HA saRNA) formulated in the interior of PE-LNP 11-65, PE-LNP 18-65 and PE-LNP 18-80, respectively to a total injection volume of 50 μL in 1×PBS and boosted with the identical formulation after 4 weeks. jetPEI/HA saRNA complexes were used as a control. Blood was collected after 4 and 6 weeks from study onset via tail bleeding. Blood was collected and centrifuged at 10,000×RPM for 5 min. The serum was harvested and stored at −80° C.

In vivo immunogenicity of SARS-CoV-2 saRNA-loaded PE-LNPs Female BALB/c mice (Charles River, UK), 6-8 weeks of age, were placed into groups of n=5. Mice were immunized IM in one hind leg quadriceps muscle with 1 μg of saRNA encoding a pre-fusion stabilized SARS-CoV-2 spike protein formulated in the interior of PE-LNP 11-65 and PE-LNP 18-65, respectively, to a total injection volume of 50 μL in 1×PBS. Blood was collected at week 4 from study onset via tail bleeding. Blood was collected and centrifuged at 10,000×RPM for 5 min. The serum was harvested and stored at −80° C.

### HA-Specific ELISA

HA antibody titers were quantitatively evaluated using the immunoglobulin ELISA protocol. Briefly, 0.5 μg mL⁻¹ of HA-coated ELISA plate was blocked with 1% (w/v) bull serum albumin (BSA)/0.05% (v/v) Tween-20 in PBS. After washing, diluted serum samples were added to the plates, incubated for 2 h, and washed, and a 1:400o dilution of anti-mouse IgG-HRP (Southern Biotech, UK) was used. Standards were prepared by coating ELISA plate wells with antimouse Kappa (1:1000) and Lambda (1:1000) light chain (Serotec, UK), blocking with PBS/1% (w/v) BSA/0.05% (v/v) Tween-20, washing, and adding purified IgG (Southern Biotech, UK) starting at 1000 ng mL-¹ and titrating down with a 5-fold dilution series. Samples and standards were developed using TMB (3,3′; 5,5′-tetramethylbenzidine), and the reaction was stopped after 5 min with Stop Solution (Insight Biotechnologies, UK). Absorbance was read on a spectrophotometer (VersaMax, Molecular Devices) with SoftMax Pro GxP v₅ software.

### SARS-CoV-2-Specific ELISA

SARS-CoV-2 antibody titers were quantitatively evaluated using the immunoglobulin ELISA protocol, following a similar procedure to the HA-specific ELISA.

### Influenza Challenge

Three weeks after the boost injection, mice were challenged with 4.2×10⁵ plaque forming units (pfu) of influenza (Cal/09) suspended in 100 μL of PBS. Mice were anesthetized using isoflurane, challenged intranasally (IN), and weighed each day to determine weight loss. According to the challenge protocol humane end-points, mice were euthanized if they sustained more than 3 days of 20% weight loss or 1 day of 25% weight loss.

### Results and Discussion

To study the in vivo saRNA expression efficiency, luciferase saRNA was formulated in various PE-LNPs and administered to mice by intramuscular injection with only one dose (5 μg/leg). After 7 days, the mice were imaged and the relative fluorescence intensity was quantified. The signal of saRNA/DOTAP lipid nanostructure-treated mice was hardly detectable, which might be due to the limited saRNA encapsulation ability and poor stability of saRNA/DOTAP lipid nanostructure. However, all the PE-LNP groups exhibited protein expression. Compared with PE-LNP 5-65, the three formulations PE-LNP 11-65, PE-LNP 18-65 and PE-LNP 18-80 showed significantly higher luciferase expression (p<0.05) (FIGS. 14A and 14B). The luciferase expression in PE-LNP 18-80 M₂ (prepared by Method 2, with saRNA on the nanoparticle surface) was significantly lower than that of PE-LNP 18-80 (prepared by Method 1, with saRNA in the nanoparticle core) (p<0.05) although they showed similar in vitro transfection efficiency (FIG. 9A). It further confirms that formulation of saRNA in the core of PE-LNPs is more favorable than formulation on the nanoparticle surface. Although the in vitro transfection efficiency of PE-LNP 18-65 was one magnitude higher than that of PE-LNP 11-65 (FIG. 8B), the in vivo luciferase expression of these two groups presented the similar signal. This might be because of the superior cell viability of PE-LNP 11-65 with a lower N/P molar ratio.

Furthermore, the inventors evaluated the immunogenicity and protective capacity of HA-encoding saRNA formulated in the interior of PE-LNPs after IM injection. One commercially available linear PEI, jetPEI which has previously been shown to effectively deliver RNA in vivo³¹, was used as a positive control. Mice received a prime and boost of 1 μg of saRNA formulated with jetPEI, PE-LNP 11-65, PE-LNP 18-65 and PE-LNP 18-80. The boost was administered 4 weeks after the initial prime. The mice were challenged IN with a Cal/09 influenza virus ² weeks after the boost and weighed daily to monitor disease pathology (FIG. 14C). A relatively high dose of 4.2×10⁵ pfu was used in order to discriminate weight loss between groups. All mice in the naive group lost>25% of their body weight after day 6 and had to be euthanized according to the challenge protocol humane endpoints, whilst encouragingly, all mice in jetPEI and PE-LNPs groups were completely protected (FIG. 14E). Mice in all three PE-LNPs groups lost less than 10% weight during the whole period of observation, especially the PE-LNP 11-65 group showing the least amount of weight loss (˜8%), which indicated lesser side effects and better protection. The HA IgG antibody titers (FIG. 14D) directly reflected the challenge results. All these three PE-LNPs groups, PE-LNP 11-65, PE-LNP 18-65 and PE-LNP 18-80, induced high antibody titers with no significant difference (˜1×10⁵ ng mL¹ after 6 weeks), which were comparable to jetPEI. This was in agreement the in vivo protein expression results shown in FIG. 14B. It indicates that all these PE-LNPs groups reached the threshold of protein expression needed to stimulate a robust immune response. In this respect, PE-LNPs provide more flexibility in formulation compositions while ensuring effectiveness and should be advantageous for the delivery of biological molecules including RNA vaccines and therapeutics.

The inventors also evaluated the immunogenicity of nCoV-encoding saRNA formulated in the interior of PE-LNPs after IM injection. Mice received a prime of 1 μg of saRNA formulated in PE-LNP 11-65 and PE-LNP 18-65, respectively. As shown in FIG. 15, both PE-LNP 11-65 and PE-LNP 18-65 induced high antibody titers after the prime with no significant difference (˜1×10⁴ ng mL-¹ after 4 weeks). This demonstrated the PE-LNP system can effectively deliver RNA vaccines to address various diseases, including the global pandemic of COVID-19 and the abovementioned influenza virus.

### Example 10—Optimization of PE-LNP Compositions for Stable Storage of the Loaded saRNA in Aqueous Solution at 4° C.

### Optimisation of Polymer Composition of PE-LNP 11-65 for Storage in Aqueous Solution at 4° C.

PEG₅ₖ-PCL₃₋₅ₖ and PEG₅ₖ-PCL₁₀ₖ(Table 1) were synthesised and characterised following the same method described above in Example 1. saRNA was formulated in the interior of PE-LNP 11-65 with the amphiphilic polymers of different molecular weights using the same method described above in Example 2. The nano-formulations were stored in aqueous solution at 4° C. Their in vitro HEK₂₉₃ cell transfection efficiencies at the saRNA dose of 1 μg mL-¹ at Day 0, 7 and 21 were then measured following the abovementioned method in Example 4.

Transfection Efficiency of Different saRNA-Loaded PE-LNPs after Storage in Aqueous Solution at 4° C.

saRNA was formulated in the interior of PE-LNP 11-65, PE-LNP 18-65 and PE-LNP 18-80 using the same method described above in Example 2. The nano-formulations were stored in aqueous solution at 4° C. Their in vitro HEK₂₉₃ cell transfection efficiencies at the saRNA dose of 1 μg mL-¹ at Month 0 and 1 and 3 were then measured following the abovementioned method in Example 4.

### Results and Discussion

As shown in FIG. 16, the fresh PE-LNP 11-65 samples comprised of PEG₅ₖ-PCL₈₋₅ₖand PEG₅ₖ-PCL₁₀ₖ (containing different hydrophobic PCL chain lengths), respectively, displayed the similar DLS size, PDI, zeta potential and transfection efficiency. However, FIG. 16D shows that the PE-LNP 11-65 comprised of PEG₅ₖ-PCL₁₀ₖ with a relatively longer PCL chain demonstrated more robust functional stability during 21-day storage at 4° C. As comparison, the functional stability of PE-LNP 11-65 comprised of PEG₅ₖ-PCL₃₋₅ₖ with a relatively shorter PCL chain was notably compromised during storage, as indicated by the significant reduction in transfection efficiency after 7 and 21 days of storage at 4° C. The difference in transfection efficiency during storage suggests that the chain length of the hydrophobic block of the amphiphilic polymer plays an important role in maintaining the RNA stability during storage.

As the PEG₅ₖ-PCL₁₀ₖ polymer was shown to be a superior option, a further investigation of the storage at 4° C. of liquid formulations of saRNA-loaded PE-LNPs without the presence of any stabilizing molecules was conducted. FIG. 17 depicts the transfection efficiencies for PE-LNP 11-65, PE-LNP 18-65 and PE-LNP 18-80 during three-month storage in aqueous solution at 4° C. While the functional stability of the saRNA-loaded PE-LNP 18-65 and PE-LNP 18-80 showed no obvious change within the first month of storage, a reduction in the transfection efficiency of PE-LNP 11-65 was observed. This result suggests that a higher N/P ratio of 18 could provide a higher level of protection for the nucleic acid payload during storage in aqueous solution, possibly due to the formation of a more compact lipid nanostructure in the PE-LNP core.

### Example 11-Stable Storage of saRNA-Loaded PE-LNPs in Aqueous Solution at Room Temperature

Transfection efficiency of saRNA-loaded PE-LNP 11-65 after storage in aqueous solution at room temperature saRNA was formulated in the interior of PE-LNP 11-65 using the same method described above in Example 2. The nano-formulation was stored in aqueous solution at room temperature. Its in vitro HEK₂₉₃ cell transfection efficiency at the saRNA dose of 1 μg mL-¹ after storage at room temperature for 21 days was then measured and compared with the freshly prepared saRNA-loaded PE-LNP 11-65 following the abovementioned method in Example 4.

Transfection Efficiency of Different saRNA-Loaded PE-LNPs after Storage in Aqueous Solution at Room Temperature

saRNA was formulated in the interior of PE-LNP 11-65, PE-LNP 18-65 and PE-LNP 18-80, respectively, using the same method described above in Example 2. The nano-formulations were stored in aqueous solution at room temperature. Their in vitro HEK₂₉₃ cell transfection efficiencies at the saRNA dose of 1 μg mL⁻¹ at Day 0, 14, 21 and 28 were then measured following the abovementioned method in Example 4.

### Results and Discussion

As shown in FIGS. 18 and 19, no decrease was observed in the transfection efficiency of saRNA formulated in the interior of PE-LNP 11-65, PE-LNP 18-65 and PE-LNP 18-80 after storage in aqueous solution at room temperature for 21 days, compared with the freshly prepared PE-LNP formulations with the equivalent saRNA dose of 1 μg mL-¹. After 28 days, high transfection efficiency was maintained, although a slight decrease was observed as compared to the fresh samples. As shown in FIGS. 19B-19D, PE-LNP 11-65, PE-LNP 18-65 and PE-LNP 18-80 also well retained the hydrodynamic size, PDI, and zeta potential during storage at room temperature. This further confirms that the polymer shell layer formed by self-assembly of the amphiphilic PEG₅k-PCL₁₀ₖ copolymer can improve the nanoparticle stability and offer optimal protection of biological payloads, in particular readily hydrolyzed/degraded RNA molecules, from the harsh external environment in liquid formulation.

### Example 12—Stable Storage of saRNA-Loaded PE-LNPs in the Presence of Trehalose in Aqueous Solution at 4° C.

Transfection Efficiency of Trehalose-Containing, saRNA-Loaded PE-LNP 11-65 after Storage in Aqueous Solution at 4° C.

The aqueous solution of saRNA (40 μg mL⁻¹) and trehalose at the trehalose/saRNA (w/w) weight ratio of 100 was employed for the preparation of saRNA- and trehalose-coloaded PE-LNP 11-65 using the similar method as described above in Example 2. Then, additional trehalose was mixed with the obtained saRNA- and trehalose-coloaded PE-LNP 11-65 for topping up the total trehalose (both interior and exterior) to 250 mg mL-¹.

As comparison, the aqueous solution of saRNA (40 μg mL⁻¹) was formulated in the interior of PE-LNP 11-65 using the same method described above in Example 2. The obtained nano-formulation was then mixed with exterior trehalose at a concentration of 250 mg mL-1.

Those nano-formulations were stored in aqueous solution at 4° C. for 383 days, and then their in vitro HEK₂₉₃ cell transfection efficiencies at the saRNA dose of 1 μg mL⁻¹ were measured and compared with the freshly prepared, saRNA-loaded, trehalose-free PE-LNP 11-65.

### Results and Discussion

RNA molecules are very fragile and can readily degrade in exposed environments, thus requiring RNA vaccines and therapeutics to be stored and transported in a very challenging cold chain. Pfizer/BioNTech's mRNA vaccine, the world's first approved COVID-19 vaccine, is plagued by the major hurdle requiring storage at −70° C. Other RNA vaccines have the similar thermal stability issues, for example, Moderna's mRNA vaccine needs to be held at −20° C. for storage. This makes it extremely challenging for RNA vaccines to reach the required speed and scale of deployment to ensure herd immunity.

PE-LNPs formulated with RNA vaccines exhibited efficient in vivo protein expression and excellent immunogenicity in Example 9 described above (FIGS. 14 and 15). The abovementioned Example 10 demonstrated that the saRNA-loaded PE-LNP formulations without any stabilizing molecules could maintain the high transfection efficiency after storage in aqueous solution at 4° C. for 3 months (FIG. 17). As demonstrated in Example 11 above, after storage in aqueous solution at room temperature for 21 days, the saRNA-loaded PE-LNP formulations showed no reduction in the transfection efficiency compared with their freshly prepared counterparts at an equivalent saRNA dose (FIGS. 18 and 19). This was due to their unique PE-LNP nanostructure offering optimal protection of RNA payloads in the nanoparticle core.

To further prolong the shelf life of the RNA nano-formulations in aqueous solution, the exterior and/or interior stabilizing molecules such as trehalose was included (FIG. 20) following the method in Example 10. As shown in FIG. 21, the saRNA- and trehalose-coloaded PE-LNP 11-65 and the saRNA-loaded PE-LNP 11-65 with mixed exterior trehalose displayed the comparable transfection efficiency, with no significant difference compared with the freshly prepared, saRNA-loaded, trehalose-free PE-LNP 11-65 after storage in aqueous solution at 4° C. for over one year (383 days).

### Example 13—Stable Storage of saRNA- and Trehalose-Coloaded PE-LNPs in Aqueous Solution at Room Temperature

Transfection Efficiency of saRNA- and Trehalose-Coloaded PE-LNPs after Storage in Aqueous Solution at Room Temperature

The aqueous solution of saRNA (40 μg mL⁻¹) and trehalose at the trehalose/saRNA (w/w) weight ratio of 100 was employed for the preparation of saRNA- and trehalose-coloaded PE-LNP 11-65, PE-LNP 16-65 and PE-LNP 18-80, followed by mixing with additional trehalose for topping up to the total trehalose at 250 mg mL-¹, using the same method as described above in Example 12.

Those nano-formulations were stored in aqueous solution at room temperature, and then their in vitro HEK₂₉₃ cell transfection efficiencies at the saRNA dose of 1 μg mL⁻¹ at Day 0, 14, 21 and 28 were measured and compared with the freshly prepared, saRNA-loaded, trehalose-free PE-LNP 11-65, PE-LNP 16-65 and PE-LNP 18-80, respectively.

### Results and Discussion

The inventors then proceed to examine the effect of trehalose as a stabilizing molecule in the aqueous solution for storage at room temperature (FIG. 20). With the fresh saRNA-loaded, trehalose-free PE-LNPs as a negative control, the saRNA- and trehalose-coloaded PE-LNPs demonstrated the comparable transfection efficiency after 4 weeks of storage at 20° C. (FIG. 22A). Compared with the negative control, the saRNA- and trehalose-coloaded PE-LNPs also well retained the DLS size (FIG. 22B), PDI (FIG. 22C) and zeta potential (FIG. 22D) during the storage at 20° C. for 4 weeks. Those suggest potential for prolonged storage.

### Example 14—Optimization of Lypholization Conditions in the Presence of Trehalose and Stable Storage of the Lyophilized saRNA-Loaded PE-LNPs at 40C

Optimization of Lyophilization Conditions for saRNA-Loaded PE-LNPs in the Presence of Exterior Trehalose

Different concentrations of saRNA (e.g., 20, 40, 60 and 100 μg mL-1) were formulated in the interior of PE-LNP 11-65 using the method described above in Example 2. The obtained nanoparticles were then mixed with exterior trehalose at a fixed concentration of 200 mg mL-¹. The formulations were frozen in a −800C freezer, lyophilized for 48 h and then immediately rehydrated with RNase-free water. The DLS particle size distribution and in vitro HEK₂₉₃ cell transfection efficiency of the rehydrated PE-LNPs at the saRNA dose of 1 μg mL⁻¹ were evaluated to optimise the saRNA concentration during freeze-drying. After that, PE-LNP 11-65 loaded with the optimized saRNA concentration but mixed with different exterior trehalose concentrations (e.g., 150, 200, 250, 300 and 400 mg mL-1) were lyophilized and immediately rehydrated with RNase-free water for further analysis by DLS and in vitro transfection. Accordingly, the optimal concentrations of the loaded saRNA and the mixed trehalose for lyophilization of PE-LNP 11-65 formulations were identified.

Storage at 4° C. of Lyophilized saRNA-Loaded PE-LNPs saRNA in the Presence of Trehalose

The saRNA-loaded PE-LNP 11-65 containing 250 mg mL⁻¹ exterior trehalose, as well as the saRNA- and trehalose-coloaded PE-LNP 11-65 (trehalose/saRNA weight ratio=100 for co-encapsulation, followed by mixing with additional trehalose for topping up the total trehalose to 250 mg mL-1) were lyophilized and then stored at 4° C. After that, the lyophilized PE-LNP formulations were rehydrated with RNase-free water for measurements of their in vitro HEK₂₉₃ cell transfection efficiencies at the saRNA dose of 1 μg mL-¹.

Optimization of Lyophilization Conditions for saRNA- and Trehalose-Coloaded PE-LNPs

The aqueous solution of saRNA (fixed at 40 μg mL⁻¹) and trehalose in RNase-free water at various trehalose/saRNA (w/w) weight ratios (e.g., 25, 50, 100, 200, 400 and 6250) was employed for the preparation of saRNA- and trehalose-coloaded PE-LNP 11-65 using the method described above in Example 2. Then, additional trehalose was mixed with the obtained saRNA- and trehalose-coloded PE-LNP 11-65 for topping up the total trehalose (both interior and exterior) to 250 mg mL-¹ which was optimized from the abovementioned experiment in Example 14. In the particular case of saRNA- and trehalose-coloaded PE-LNP 11-65 with the trehalose/saRNA (w/w) ratio at 6250 and the saRNA concentration at 40 μg mL⁻¹, there was no need to add additional trehalose for topping up. After lyophilization and immediate rehydration with RNase-free water, the colloidal stability of the saRNA- and trehalose-coloaded PE-LNP 11-65 was tested by DLS and their in vitro transfection efficiency at the saRNA dose of 1 μg mL⁻¹ was analysed.

### Results and Discussion

Another strategy that the inventors have utilized for stable storage of RNA vaccines and therapeutics without the need for a very challenging cold chain is lyophilization of RNA-loaded PE-LNPs in the presence of exterior and/or interior stabilizing molecules such as trehalose (FIG. 20). Lyophilization processes can potentially severely reduce colloidal stability of RNA delivery nano-formulations, leading to an irreversibly decreased efficacy. It has been reported by other researchers that the colloidal stability of nanoparticles can be improved by simply mixing trehalose outside the nanoparticles during freeze-drying^{2l}, because the vitrification of trehalose can immobilize the nanoparticles in a rigid, amorphous glassy sugar matrix and by doing so dramatically decrease the aggregation or rupture of the nanoparticles. Furthermore, as the PE-LNPs have a unique structure which contains interior hydrophilic domains, trehalose could also be coloaded with RNA in the interior of PE-LNPs and make direct interactions with RNA. The hydroxyl groups of trehalose can form hydrogen bonds with RNA molecules, thereby minimizing hydration of RNA during lyophilization. Therefore, the thermal stability of RNA could be enhanced remarkably.

First, PE-LNP 11-65 containing a fixed concentration of exterior trehalose at 200 mg mL⁻¹ and various concentrations (20-100 μg mL⁻¹) of saRNA loaded in the nanoparticle interior were lyophilized and immediately rehydrated with RNase-free water. The transfection efficiency of the rehydrated PE-LNP 11-65 were then evaluated. Interestingly, FIG. 23 displays the synergistic effect of exterior trehalose on the HEK₂₉₃ cell transfection, showing an enhancement in transfection efficiency of all the lyophilized PE-LNP 11-65 formulations by 1-1.5 orders of magnitude compared with the freshly prepared trehalose-free PE-LNP 11-65 at the equivalent saRNA dose of 1 μg mL-¹. According to FIG. 23, 40 μg mL⁻¹ was chosen as the optimal saRNA concentration for lyophilization of saRNA-loaded PE-LNP 11-65 in the presence of 200 mg mL-¹ exterior trehalose.

Further, PE-LNP 11-65 containing the fixed interior saRNA concentration (40 μg mL-¹) and various concentrations of exterior trehalose were lyophilized and immediately rehydrated with RNase-free water. According to FIG. 24, 250 mg mL-¹ was chosen as the optimal exterior trehalose concentration for lyophilization of PE-LNP 11-65 loaded with 40 μg mL-¹ interior saRNA. As shown in FIG. 24, the lyophilized formulation under this condition, upon immediate rehydration, showed the highest transfection efficiency, which was one order of magnitude higher than the freshly prepared trehalose-free PE-LNP 11-65 at the equivalent saRNA dose of 1 μg mL-1.

FIG. 25 shows stable RNA storage at 4° C. after lyophilization of the PE-LNP 11-65 formulation containing 40 μg mL-¹ interior saRNA and 250 mg mL-¹ exterior trehalose. After storage at 4° C. for 28 days and then rehydration with RNase-free water, no reduction in transfection efficiency of the lyophilized formulation was observed compared with the freshly prepared trehalose-free PE-LNP 11-65 at the equivalent saRNA dose of 1 μg mL-¹. This suggests that the combined effects of exterior trehalose with inherent protection offered by the core-shell PE-LNP nanostructure well retained the RNA stability during storage at 4° C.

In order to further improve the RNA stability during storage, the inventors prepared the PE-LNP 11-65 coloaded with saRNA (at the fixed concentration of 40 μg mL-¹) and trehalose (at different trehalose/saRNA weight ratios) in the hydrophilic core of the nanoparticles.

Additional trehalose was then mixed with the resulting saRNA- and trehalose-coloaded PE-LNP 11-65 for topping up the total trehalose (both interior and exterior) to 250 mg mL-¹, followed by lyophilization of the formulations. It was found that when the trehalose/saRNA (w/w) weight ratio was above 400, the size of nanoparticles after lyophilization and rehydration with RNase-free water became bigger than 300 nm. The trehalose/saRNA weight ratio of 100 was the most favorable, leading to formation of the rehydrated PE-LNP 11-65 with the small uniform DLS size of 167.7 f 3.6 nm and the good monodispersity with a PDI of 0.313±0.017, comparable to the freshly prepared trehalose-free PE-LNP 11-65 (size=131.3±0.5 nm and PDI=0.265, shown in FIG. 2C). This suggests that existence of both interior and exterior trehalose well retained the colloidal stability of PE-LNP 11-65 during the lyophilization and rehydration processes. In addition, saRNA- and trehalose-coloaded PE-LNP 11-65 showed a high saRNA encapsulation efficiency at 85.2 f 4.8%, which was comparable with that of trehalose-free saRNA-loaded PE-LNP 11-65 (93.6 f 3.2%).

Although the total trehalose concentration was fixed at 250 mg mL-¹, it was favorable to increase the trehalose/saRNA (w/w) weight ratio up to 50-100 (i.e., increasing the proportion of the interior trehalose) for lyophilization of PE-LNP 11-65 formulations. As shown in FIG. 26, the saRNA- and trehalose-coloaded PE-LNP 11-65 formulations lyophilized under this condition, upon immediate rehydration with RNase-free water, demonstrated dramatically improved transfection efficiency, which was 1.5 orders of magnitude higher than the freshly prepared trehalose-free PE-LNP 11-65 at the equivalent saRNA dose of 1 μg mL-¹. A further increase in the trehalose/saRNA (w/w) weight ratio above 100 led to the significantly decreased transfection efficiency although it was still one order of magnitude higher than the freshly prepared trehalose-free PE-LNP 11-65. This suggests that the co-existence of saRNA and trehalose in the nanoparticle interior, combined with the presence of exterior trehalose, can exert the synergistic effect and considerably improve the RNA stability and efficacy.

FIG. 27 compares the stability of the lyophilized PE-LNP 11-65 with and without interior trehalose. After storage at 4° C. for over one year (380 days) and then rehydration with RNase-free water, saRNA- and trehalose-coloaded PE-LNP 11-65 showed the significantly higher transfection efficiency (p<0.05) than that of the trehalose-free fresh samples. This could be explained as trehalose, in addition to stabilizing the formulation, provided the synergistic effect on improving transfection. The formulation without interior trehalose showed a loss of transfection efficiency by more than 2 magnitudes after storage at 4° C. for over one year. This demonstrates that the PE-LNP optimised with the presence of both interior and exterior trehalose can effectively maintain their functional stability of the lyophilized formulations during long-term storage.

The optimized lyophilization conditions for saRNA- and trehalose-coloaded PE-LNP 11-65 (40 μg mL-¹ saRNA and trehalose/saRNA weight ratio=100 for co-encapsulation, followed by mixing with additional trehalose for topping up the total trehalose to 250 mg mL-1) was chosen for the further heat burden study of the lyophilized formulations.

### Example 15— Heat Burden Study of Lyophilized saRNA- and Trehalose-Coloaded PE-LNPs for Stable Storage at 40° C.

The optimized saRNA- and trehalose-coloaded PE-LNP 11-65 formulations were lyophilized and then held for storage at 40° C. (tropical conditions). The saRNA-loaded PE-LNP 11-65 mixed with exterior trehalose was lyophilized as a control. After storage after a certain period, the lyophilized PE-LNP 11-65 formulations were rehydrated with RNase-free water and their in vitro transfection efficiencies were measured.

### Results and Discussion

To evaluate the potential of RNA- and trehalose-coloaded PE-LNPs for non-cold chain storage, the optimized saRNA- and trehalose-coloaded PE-LNP 11-65 were lyophilized and held for storage at 40° C. for 1, 3, 5, 7 or 14 days, respectively. FIG. 28 shows that, after lyophilization and then immediate rehydration with RNase-free water (Day 0), the transfection efficiencies of saRNA- and trehalose-coloaded PE-LNP 11-65 and saRNA-loaded PE-LNP 11-65 mixed with exterior trehalose were both ˜1.5 orders of magnitude higher than the freshly prepared trehalose-free PE-LNP 11-65, further confirming the synergistic effect of trehalose. As shown in FIGS. 31 and 32, even after being held for storage at the high temperature of 40° C. for 14 days, no reduction in the transfection efficiency of saRNA- and trehalose-coloaded PE-LNP 11-65 or saRNA-loaded PE-LNP 11-65 mixed with exterior trehalose was observed compared with the freshly prepared trehalose-free saRNA-loaded PE-LNP11-65, indicative of the excellent thermal stability and efficacy of RNA at room temperatures and even under the tropical conditions.

The inventors then utilized ultrafiltration centrifugation to remove free (unloaded) exterior trehalose after lyophilization, storage at 40° C. for 7 days and rehydration of saRNA- and trehalose-coloaded PE-LNP 11-65 and saRNA-loaded PE-LNP 11-65 mixed with exterior trehalose, respectively. FIG. 30 shows that, after removal of exterior trehalose, the transfection efficiencies of the rehydrated PE-LNP 11-65 without interior trehalose considerably decreased compared to their counterparts without ultrafiltration centrifugation, especially on Day 7 of storage at 40° C. As comparison, it was interesting to note that, after removal of exterior trehalose, the transfection efficiency of the rehydrated PE-LNP 11-65 coloaded with saRNA and trehalose in the hydrophilic core of the nanoparticles was comparable to the freshly prepared trehalose-free saRNA-loaded PE-LNP 11-65 after storage at 40° C. for 7 days. These results further confirm the critical role of the interior trehalose on enhancement of the thermal stability and efficacy of RNA coloaded in the PE-LNPs.

### Example 16—Effect of Trehalose on PE-LNP Mediated Intracellular Delivery of Different RNA Molecules

Using the method described above in Example 12, trehalose and 50 μg mL⁻¹ calcein were pre-dissolved in the 40 μg mL⁻¹ saRNA solution in RNase-free deionized water at the trehalose/saRNA (w/w) weight ratio of 100 for preparing saRNA-, trehalose- and calcein-coloaded PE-LNP 11-65, to which additional trehalose was mixed for topping up to the total trehalose (both interior and exterior) at 250 mg mL-¹. saRNA- and calcein-coloaded PE-LNP 11-65 (trehalose-free) were prepared as a control. The uptake and intracellular trafficking of the two calcein-containing PE-LNP 11-65 formulations were investigated by laser scanning confocal microscopy. A 2 mL amount of HEK₂₉₃ cells (2×10⁵ cells per dish) was seeded into a 35 mm glass-bottom culture dish and cultured for 24 h. saRNA-, trehalose- and calcein-coloaded PE-LNP 11-65, or saRNA- and calcein-coloaded PE-LNP 11-65 were added to the culture dish (2 μg saRNA per dish). After treatment for 2 h, cells were washed and replenished with complete medium for a further 4 h of incubation. Cells were imaged using a Leica SP8 Inverted confocal microscope and mean fluorescence intensities of calcein in the confocal microscopy images were analyzed by ImageJ.

GFP-expressing mRNA- and trehalose-coloaded PE-LNP 11-65 (trehalose/mRNA weight ratio=100 for co-encapsulation, followed by mixing withb additional trehalose for topping up to the total trehalose at 10 mg mL¹) were also prepared using the method described above in Example 12. Trehalose-free mRNA-loaded PE-LNP 11-65 were prepared as a control. HEK₂₉₃ cells were seeded in a 6-well plate at 5×10⁵/well and cultured for 48 h, and the in vitro transfection was quantitatively analysed by flow cytometry (Canto, BD, USA).

### Results and Discussion

The considerably enhanced transfection efficiencies of the saRNA-loaded PE-LNPs due to the presence of exterior/interior trehalose, as demonstrated in FIGS. 23-30, has prompted the inventors to further investigate the synergistic effect of the added trehalose. The membrane-impermeable dye, calcein, was coloaded in the interior of the two PE-LNP 11-65 formulations as an indicator of cellular uptake and intracellular trafficking. As shown in the confocal microscopy images (FIG. 31A), HEK₂₉₃ cells treated with the freshly prepared saRNA-, trehalose and calcein-coloaded PE-LNP 11-65 showed the considerably stronger green diffuse staining throughout the cells compared to those treated with the freshly prepared saRNA- and calcein-coloaded PE-LNP 11-65. The mean florescence intensity (MFI) was analysed by ImageJ. FIG. 31B shows the cells treated with saRNA-, trehalose and calcein-coloaded PE-LNP 11-65 had the significantly higher MFI compared with those treated with saRNA- and calcein-coloaded PE-LNP 11-65. This validated that the presence of trehalose could enhance the cellular uptake and subsequent endosomal escape.

The effect of trehalose on intracellular delivery of GFP-encoding mRNA to HEK₂₉₃ cells by PE-LNP 11-65 was also investigated. As shown in FIG. 32, after treatment with the freshly prepared mRNA- and trehalose-coloaded PE-LNP 11-65, the percentage of GFP-expressing HEK₂₉₃ cells was increased from 51.9±4.9% to 74.2 f 2.2% and the MFI was increased by 1.7-fold as compared to the freshly prepared trehalose-free mRNA-loaded PE-LNP 11-65. This further confirms the synergetic effect of trehalose on intracellular delivery of various nucleic acids by the PE-LNP based formulations.

### Example 17 Variation in the Lipid Compositions of PE-LNPs

saRNA was formulated in the interior of PE-LNP 11-65 in presence of cholesterol following a similar method as described above in Example 2, where cholesterol was co-dissolved in THF with DOTAP and PEG-PCL. The cholesterol content (wt %) was defined as the weight percentage of cholesterol relative to cholesterol and DOTAP. The size, PDI and zeta potential of the nanoformulations were evaluated using a Litesizer (Anton Paar, UK). Their in vitro HEK₂₉₃ cell transfection efficiencies at the saRNA dose of 1 μg mL¹ were measured following the abovementioned method in Example 4.

### Results and Discussion

The preparation method for the PE-LNP system is readily adaptable to incorporate various lipids and their combinations to the nanoparticle core, including but not limited to cationic/ionizable lipids with chargeable groups and sterols such as cholesterol. The inventors chose cholesterol, to be formulated in the PE-LNP system, to further demonstrate the versatility of the nano system in its lipid compositions.

FIG. 33 shows the physicochemical characterisation of PE-LNP 11′-65′ and the transfection efficiency of saRNA-loaded PE-LNP 11-65 formulated with cholesterol. The PE-LNP 11′-65′ in the presence of cholesterol showed size, PDI and zeta potential that are suitable for biomolecule delivery. The inventors then proceeded to investigate the effect of cholesterol content on the transfection efficiency. When the cholesterol content was increased from 20 wt % to 40 wt %, a significant increase in the transfection efficiency by more than one magnitude was observed. This demonstrates the PE-LNP system is compatible with various lipid molecules and their combinations. The transfection efficiency of the nanoformulation can be tailored for different applications by adjusting the lipid compositions.

### CONCLUSIONS

The inventors have developed polymer-enveloped lipid nanoparticles (PE-LNPs) to achieve efficient intracellular delivery of biological molecules including RNA both in vitro and in vivo and enable stable storage of vaccines and therapeutics without the need for a cold chain. This nano-formulation platform has the characteristics of an ideal carrier, including the favorable safety profile, compact size, controlled charge, high loading efficiency, efficient endosomolytic activity, superior colloidal and payload (RNA) stability, as well as simple, cost-effective and readily scalable preparation method.

PE-LNPs are composed of two main structural components: amphiphilic polymers such as PEG-PCL and cationic or ionizable lipids such as DOTAP, which have been proven to be biocompatible and approved by FDA. PE-LNPs can be prepared using a simple one-pot method through easy and rapid mixing and organic solvent evaporation. Cryo-TEM and FRET analysis have shown that the interior structure of PE-LNPs consists of lipid nanostructures, which are favorable for efficient payload encapsulation. The self-assembled PEG-PCL outer layer surrounding the interior lipid nanostructures can ensure colloidal stability and serum compatibility, and well protect the functionality of payloads such as extremely unstable RNA. PE-LNPs exhibited the excellent stability and high in vitro transfection efficiency, which can be four orders of magnitude higher than the commercially available PEI in the presence of FBS.

Mice transfected by IM injection with PE-LNP 11-65, PE-LNP 18-65 and PE-LNP 18-80, respectively showed strong luciferase expression. Furthermore, all mice were effectively protected in the Cal/09 influenza challenge after immunization with HA saRNA formulated in the interior of PE-LNP 11-65, PE-LNP 18-65 and PE-LNP 18-80, respectively because all of the nano-formulations induced high HA IgG antibody titers. The average weight loss of the mice immunized with HA saRNA-loaded PE-LNPs was less than 10%, especially PE-LNP 11-65 inducing the least amount of weight loss (˜8%).

The inventors have demonstrated two strategies for stable storage of biological payloads, in particular readily hydrolyzed/degraded RNA molecules, at ambient temperatures. The optimal protection offered by the core-shell nanoparticle structure enabled stable storage of RNA-loaded PE-LNPs in aqueous solution at room temperature. Another strategy for stable RNA storage is lyophilization of RNA-loaded PE-LNPs in the presence of exterior and/or interior stabilizing molecules such as trehalose. saRNA- and trehalose-coloaded PE-LNPs and saRNA-loaded PE-LNPs mixed with exterior trehalose showed the desired thermal stability of saRNA after lyophilization, storage at ambient temperatures as high as 40° C. (tropical conditions), and rehydration, with the former showing better performance. These demonstrate that the PE-LNP nano-formulations can enable stable storage of vaccines and therapeutics at ambient temperatures without the need for a cold chain. This offers a viable solution to improving global distribution of vaccine and therapeutic formulations.

Furthermore, the PE-LNPs developed by the inventors have been demonstrated to be generalizable to efficient intracellular delivery to different cell types and non-cold chain storage of various vaccines and therapeutics (based on biological molecules including but not limited to saRNA and mRNA).

### Materials

mPEG-OH (Mₙ=5,000 and 2,000), ε-caprolactone (ε-CL), toluene, diethyl ether, tetrahydrofuran (THF), 4% paraformaldehyde solution, fluorescein isothiocyanate (FITC), Hoechst, LysoTracker (red), Triton X-10o, bull serum albumin (BSA) and Tween-20 were purchased from Sigma-Aldrich. 1,2-dioleoyl-3-trimethylammonium-propane (DOTAP, chloride salt) was bought from Avanti Polar Lipids. Trypsin-EDTA (0.25%, w/v), fetal bovine serum (FBS) and 1% penicillin/streptomycin were bought from Gibco (CA, USA). ONE-Glo™ Luciferase Assay System was obtained from InvivoGen. Phosphate buffered saline (PBS) and DMEM medium were obtained from Hyclone Laboratories (UT, USA). RNase-free water, RNase-free PBS (10×) and TPCK-trypsin were purchased from Thermo Fisher Scientific (UK). Trehalose Assay Kit was purchased from Abbexa. XenoLight RediJect D-Luciferin Substrate was bought from Perkin Elmer. Firefly luciferase saRNA and saRNA that encodes the H₁ hemagglutinin of the Cal/09 virus were both kindly gifted by Prof. Robin Shattock's group at St Mary Hospital and Department of Infectious Disease, Imperial College London.

### Cell Culture

HEK₂₉₃ (human embryonic kidney cell line) cells and HeLa (human cervical cancer cell line) cells were obtained from the ATCC (American Type Culture Collection, Wesel, Germany) and were cultured in high glucose DMEM medium (Gibco) supplemented with 10% (v/v) FBS (Gibco) and 1% (v/v) penicillin/streptomycin (Gibco). Jurkat human T lymphocyte cells (Clone E6-1, ATCC® TIB-152™) were cultured in either RPMI-1640 or OPTIMEM medium supplemented with 10% (v/v) FBS, 100 U mL¹ penicillin, 100 μg mL¹ streptavidin and 2 mM L-glutamine (Rio medium). Cells were incubated in a humidified incubator with 5% CO₂ at 37° C.

### REFERENCES

1. Petsch, B.; Schnee, M.; Vogel, A. B.; Lange, E.; Hoffmann, B.; Voss, D.; Schlake, T.; Thess, A.; Kallen, K. J.; Stitz, L.; Kramps, T., Protective efficacy of in vitro synthesized, specific mRNA vaccines against influenza A virus infection. Nature biotechnology 2012, 30 (12), 1210-6.
2. Schnee, M.; Vogel, A. B.; Voss, D.; Petsch, B.; Baumhof, P.; Kramps, T.; Stitz, L., An mRNA Vaccine Encoding Rabies Virus Glycoprotein Induces Protection against Lethal Infection in Mice and Correlates of Protection in Adult and Newborn Pigs. PLoS Negl Trop Dis 2016, 10 (6), eo004746.
3. Bogers, W. M.; Oostermeijer, H.; Mooij, P.; Koopman, G.; Verschoor, E. J.; Davis, D.; Ulmer, J. B.; Brito, L. A.; Cu, Y.; Banerjee, K.; Otten, G. R.; Burke, B.; Dey, A.; Heeney, J. L.; Shen, X.; Tomaras, G. D.; Labranche, C.; Montefiori, D. C.; Liao, H. X.; Haynes, B.; Geall, A. J.; Barnett, S. W., Potent immune responses in rhesus macaques induced by nonviral delivery of a self-amplifying RNA vaccine expressing HIV type 1 envelope with a cationic nanoemulsion. J Infect Dis 2015, 211 (6), 947-55.
4. Pardi, N.; Hogan, M. J.; Pelc, R. S.; Muramatsu, H.; Andersen, H.; DeMaso, C. R.; Dowd, K. A.; Sutherland, L. L.; Scearce, R. M.; Parks, R.; Wagner, W.; Granados, A.; Greenhouse, J.; Walker, M.; Willis, E.; Yu, J. S.; McGee, C. E.; Sempowski, G. D.; Mui, B. L.; Tam, Y. K.; Huang, Y. J.; Vanlandingham, D.; Holmes, V. M.; Balachandran, H.; Sahu, S.; Lifton, M.; Higgs, S.; Hensley, S. E.; Madden, T. D.; Hope, M. J.; Kariko, K.; Santra, S.; Graham, B. S.; Lewis, M. G.; Pierson, T. C.; Haynes, B. F.; Weissman, D., Zika virus protection by a single low-dose nucleoside-modified mRNA vaccination. Nature 2017,543 (7644), 248-251.
5. Chahal, J. S.; Khan, O. F.; Cooper, C. L.; McPartlan, J. S.; Tsosie, J. K.; Tilley, L. D.; Sidik, S. M.; Lourido, S.; Langer, R.; Bavari, S.; Ploegh, H. L.; Anderson, D. G., Dendrimer-RNA nanoparticles generate protective immunity against lethal Ebola, HiNi influenza, and Toxoplasma gondii challenges with a single dose. Proceedings of the National Academy of Sciences of the United States of America 2016, 113 (29), E4133-42.
6. Sebastian, M.; Papachristofilou, A.; Weiss, C.; Fruh, M.; Cathomas, R.; Hilbe, W.; Wehler, T.; Rippin, G.; Koch, S. D.; Scheel, B.; Fotin-Mleczek, M.; Heidenreich, R.; Kallen, K. J.; Gnad-Vogt, U.; Zippelius, A., Phase Ib study evaluating a self-adjuvanted mRNA cancer vaccine (RNActive®) combined with local radiation as consolidation and maintenance treatment for patients with stage IV non-small cell lung cancer. BMC cancer 2014,14, 748.
7. Kallen, K. J.; Heidenreich, R.; Schnee, M.; Petsch, B.; Schlake, T.; Thess, A.; Baumhof, P.; Scheel, B.; Koch, S. D.; Fotin-Mleczek, M., A novel, disruptive vaccination technology: self-adjuvanted RNActive(®) vaccines. Hum Vaccin Immunother 2013, 9 (10), 2263-76.
8. Uchida, S.; Kinoh, H.; Ishii, T.; Matsui, A.; Tockary, T. A.; Takeda, K. M.; Uchida, H.; Osada, K.; Itaka, K.; Kataoka, K., Systemic delivery of messenger RNA for the treatment of pancreatic cancer using polyplex nanomicelles with a cholesterol moiety. Biomaterials 2016, 82, 221-228.
9. Oberli, M. A.; Reichmuth, A. M.; Dorkin, J. R.; Mitchell, M. J.; Fenton, O. S.; Jaklenec, A.; Anderson, D. G.; Langer, R.; Blankschtein, D., Lipid Nanoparticle Assisted mRNA Delivery for Potent Cancer Immunotherapy. Nano Lett 2017, 17 (3), 1326-1335.
10. Pardi, N.; Hogan, M. J.; Porter, F. W.; Weissman, D., mRNA vaccines—a new era in vaccinology. Nature Reviews Drug Discovery 2018, 17 (4), 261-279.
11. Fleeton, M. N.; Chen, M.; Berglund, P.; Rhodes, G.; Parker, S. E.; Murphy, M.; Atkins, G. J.; Liljestrom, P., Self-replicative RNA vaccines elicit protection against influenza A virus, respiratory syncytial virus, and a tickborne encephalitis virus. J Infect Dis 2001, 183 (9), 1395-8.
12. Martinon, F.; Krishnan, S.; Lenzen, G.; Magne, R.; Gomard, E.; Guillet, J. G.; Levy, J. P.; Meulien, P., Induction of virus-specific cytotoxic T lymphocytes in vivo by liposome-entrapped mRNA. Eur J Immunol 1993,23 (7), 1719-22.
13. Michel, T.; Luft, D.; Abraham, M. K.; Reinhardt, S.; Salinas Medina, M. L.; Kurz, J.; Schaller, M.; Avci-Adali, M.; Schlensak, C.; Peter, K.; Wendel, H. P.; Wang, X.; Krajewski, S., Cationic Nanoliposomes Meet mRNA: Efficient Delivery of Modified mRNA Using Hemocompatible and Stable Vectors for Therapeutic Applications. Mol Ther Nucleic Acids 2017, 8,459-468.
14. McKinlay, C. J.; Benner, N. L.; Haabeth, O. A.; Waymouth, R. M.; Wender, P. A., Enhanced mRNA delivery into lymphocytes enabled by lipid-varied libraries of charge-altering releasable transporters. Proceedings of the National Academy of Sciences of the United States of America 2018, 115 (26), E5859-E5866.
15. Zou, Y.; Zheng, M.; Yang, W.; Meng, F.; Miyata, K.; Kim, H. J.; Kataoka, K.; Zhong, Z., Virus-Mimicking Chimaeric Polymersomes Boost Targeted Cancer siRNA Therapy In Vivo. Adv Mater 2017, 29 (42).
16. Kim, Y.; Tewari, M.; Pajerowski, J. D.; Cai, S.; Sen, S.; Williams, J. H.; Sirsi, S. R.; Lutz, G. J.; Discher, D. E., Polymersome delivery of siRNA and antisense oligonucleotides. Journal of controlled release: official journal of the Controlled Release Society 2009, 134 (2), 132-40.
17. Islam, M. A.; Xu, Y.; Tao, W.; Ubellacker, J. M.; Lim, M.; Aum, D.; Lee, G. Y.; Zhou, K.; Zope, H.; Yu, M.; Cao, W.; Oswald, J. T.; Dinarvand, M.; Mahmoudi, M.; Langer, R.; Kantoff, P. W.; Farokhzad, O. C.; Zetter, B. R.; Shi, J., Restoration of tumour-growth suppression in vivo via systemic nanoparticle-mediated delivery of PTEN mRNA. Nat Biomed Eng 2018, 2 (11), 850-864.
18. Guan, S.; Munder, A.; Hedtfeld, S.; Braubach, P.; Glage, S.; Zhang, L.; Lienenklaus, S.; Schultze, A.; Hasenpusch, G.; Garrels, W.; Stanke, F.; Miskey, C.; Johler, S. M.; Kumar, Y.; Tummler, B.; Rudolph, C.; Ivics, Z.; Rosenecker, J., Self-assembled peptide-poloxamine nanoparticles enable in vitro and in vivo genome restoration for cystic fibrosis. Nature nanotechnology 2019, 14 (3), 287-297.
19. Sizovs, A.; Xue, L.; Tolstyka, Z. P.; Ingle, N. P.; Wu, Y.; Cortez, M.; Reineke, T. M., Poly(trehalose): sugar-coated nanocomplexes promote stabilization and effective polyplex-mediated siRNA delivery. Journal of the American Chemical Society 2013, 135 (41), 15417-24.
20. Jones, K. L.; Drane, D.; Gowans, E. J., Long-term storage of DNA-free RNA for use in vaccine studies. Biotechniques 2007, 43 (5), 675-681.
21. Zhao, P.; Hou, X.; Yan, J.; Du, S.; Xue, Y.; Li, W.; Xiang, G.; Dong, Y., Long-term storage of lipid-like nanoparticles for mRNA delivery. Bioact Mater 2020, 5 (2), 358-363.
22. Shuai, X.; Ai, H.; Nasongkla, N.; Kim, S.; Gao, J., Micellar carriers based on block copolymers of poly(epsilon-caprolactone) and poly(ethylene glycol) for doxorubicin delivery. Journal of controlled release: official journal of the Controlled Release Society 2004, 98 (3), 415-26.
23. LoPresti, C.; Lomas, H.; Massignani, M.; Smart, T.; Battaglia, G., Polymersomes: nature inspired nanometer sized compartments. Journal of Materials Chemistry 2009, 19 (22).
24. Mai, Y.; Eisenberg, A., Self-assembly of block copolymers. Chem Soc Rev 2012, 41 (18), 5969-85.
25. Lee, J. S.; Feijen, J., Polymersomes for drug delivery: design, formation and characterization. Journal of controlled release: official journal of the Controlled Release Society 2012, 161 (2), 473-83.
26. Kulkarni, C. V., Lipid crystallization: from self-assembly to hierarchical and biological ordering. Nanoscale 2012, 4 (19), 5779-91.
27. Chemin, M.; Brun, P. M.; Lecommandoux, S.; Sandre, O.; Le Meins, J. F., Hybrid polymer/lipid vesicles: fine control of the lipid and polymer distribution in the binary membrane. Soft Matter 2012, 8 (10), 2867-2874.
28. Dao, T. P. T.; Fernandes, F.; Er-Rafik, M.; Salva, R.; Schmutz, M.; Brulet, A.; Prieto, M.; Sandre, O.; Le Meins, J. F., Phase Separation and Nanodomain Formation in Hybrid Polymer/Lipid Vesicles. Acs Macro Letters 2015, 4 (2), 182-186.
29. Rahman, M. M.; Ueda, M.; Hirose, T.; Ito, Y., Spontaneous Formation of Gating Lipid Domain in Uniform-Size Peptide Vesicles for Controlled Release. Journal of the American Chemical Society 2018, 140 (51), 17956-17961.
30. Qiu, C.; Han, H. H.; Sun, J.; Zhang, H. T.; Wei, W.; Cui, S. H.; Chen, X.; Wang, J.

C.; Zhang, Q., Regulating intracellular fate of siRNA by endoplasmic reticulum membrane-decorated hybrid nanoplexes. Nat Commun 2019, 10 (1), 2702.
31. Kaczmarek, J. C.; Patel, A. K.; Kauffman, K. J.; Fenton, O. S.; Webber, M. J.; Heartlein, M. W.; DeRosa, F.; Anderson, D. G., Polymer-Lipid Nanoparticles for Systemic Delivery of mRNA to the Lungs. Angewandte Chemie 2016, 55 (44), 13808-13812.

1. A sub-micron particle comprising a payload molecule and a lipid structure being surrounded by an outer layer comprising an amphiphilic copolymer.

2. The sub-micron particle of claim 1, wherein the sub-micron particle has a largest maximum dimension of less than 1 μm.

3. The sub-micron particle of claim 1, or claim 2, wherein the payload molecule is a biomolecule and/or an active pharmaceutical ingredient (API).

4. The sub-micron particle of claim 3, wherein the biomolecule is a nucleic acid, and the nucleic acid is DNA, RNA or a DNA/RNA hybrid sequence.

5. The sub-micron particle of claim 4, wherein the RNA is self-amplifying RNA (saRNA) or messenger RNA (mRNA).

6. The sub-micron particle of any preceding claim, wherein the sub-micron particle comprises a plurality of lipid structures.

7. The sub-micron particle of any preceding claim, wherein the lipid structure is a lipid nanoparticle or a liposome, and preferably is a lipid nanoparticle.

8. The sub-micron particle of any preceding claim, wherein lipid structure comprises a cationic or ionizable lipid.

9. The sub-micron particle of any preceding claim, wherein the sub-micron particle has an N/P molar ratio of between 1:50 and 1,000:1, between 1:10 and 500:1, between 1:5 and 250:1, between 1:2 and 100:1, between 1:1 and 50:1, between 2:1 and 40:1, between 5:1 and 30:1, between 8:1 and 28:1, between 10:1 and 26:1, between 12:1 and 24:1, between 14:1 and 22:1 or between 16:1 and 20:1.

10. The sub-micron particle of any preceding claim, wherein the weight ratio of the amphiphilic copolymer to the payload molecule is between 5:1 and 1000:1, between 10:1 and 500:1, between 20:1 and 250:1, between 30:1 and 200:1, between 40:1 and 150:1, between 50:1 and 125:1, between 55:1 and 100:1, or between 60:1 and 85:1.

11. The sub-micron particle of any preceding claim, wherein the amphiphilic copolymer comprises at least one hydrophilic portion and at least one hydrophobic portion, and the hydrophilic portion comprises between 5 and 60 wt % of the amphiphilic copolymer and the hydrophobic portion comprises between 40 and 95% of the amphiphilic copolymer.

12. The sub-micron particle of any preceding claim, wherein the amphiphilic copolymer may have a molecular weight of between 1,000 and 100,000 Da.

13. The sub-micron particle of any preceding claim, wherein the weight ratio of the amphiphilic copolymer to the cationic or ionizable lipid is between 1:10 and 50:1, between 1:8 and 20:1, between 1:6 and 15:1, between 1:4 and 10:1, between 1:2 and 8:1, between 1:1.5 and 6:1, between 1:1 and 5.5:1, between 1.5:1 and 5:1, between 1.75:1 and 4.5:1, between 2:1 and 4:1, between 2.2:1 and 3.5:1, between 2.4:1 and 3:1, or between 2.5:1 and 2.7:1.

14. The sub-micron particle of any preceding claim, wherein the sub-micron particle further comprise at least one stabilizing molecule.

15. The sub-micron particle of claim 14, wherein at least one stabilizing molecule may be surrounded by the outer layer comprising the amphiphilic copolymer.

16. The sub-micron particle of claim 14, or claim 15, wherein the weight ratio of the at least one stabilizing molecule to the payload molecule is between 1:1 and 100,000:1, between 2:1 and 50,000:1, between 4:1 and 10,000:1, between 6:1 and 5,000:1, between 8:1 and 1,000:1, between 10:1 and 500:1, between 20:1 and 400:1, between 30:1 and 350:1 or between 40:1 and 300:1.

17. The sub-micron particle of any one of claims 14 to 16, wherein at least one stabilizing molecule may be disposed outside the outer layer comprising the amphiphilic copolymer, preferably wherein the at least one stabilizing molecule disposed outside the outer layer comprising the amphiphilic copolymer is disposed at a concentration of between 1 and 100,000 mg/ml, between 5 and 50,000 mg/ml, between 10 and 10,000 mg/ml, between 25 and 5,000 mg/ml, between 50 and 1,000 mg/ml, between 100 and 750 mg/ml, between 150 and 500 nm/ml, between 200 and 300 nm/ml, between 220 and 280 nm/ml, or between 240 and 260 mg/ml.

18. The sub-micron particle of any one of claims 14 to 17, wherein the or each stabilizing molecules is a carbohydrate and/or a polyol, preferably wherein the carbohydrate is a monosaccharide, a disaccharide, a trisaccharide, a polysaccharide, starch, cellulose or a polyol, and preferably is trehalose or a pharmaceutically acceptable complex, salt, solvate, tautomeric form, stereoisomer or polymorphic form thereof.

19. A method of producing a sub-micron particle, the method comprising contacting a payload molecule, a cationic or ionizable lipid, and an amphiphilic copolymer to produce the sub-micron particle.

20. The method of claim 19, wherein the method comprises:
providing a first solution comprising the cationic or ionizable lipid and the amphiphilic copolymer and an organic solvent;
providing a second solution comprising the payload molecule and water;
combining the first and second solutions to produce a reaction mixture, and thereby contacting the payload molecule, the cationic or ionizable lipid, and the amphiphilic copolymer.

21. The method of claim 19, or claim 20, wherein contacting the payload molecule, the cationic or ionizable lipid and the amphiphilic copolymer simultaneously, comprises contacting the payload molecule, the cationic or ionizable lipid, the amphiphilic copolymer and at least one stabilizing molecule.

22. The method of any one of claims 19 to 21, wherein the method comprises contacting the resultant sub-micron particle and at least one stabilizing molecule.

23. A sub-micron particle obtained or obtainable by the method any one of claim 19 to 22.

24. A pharmaceutical composition comprising the sub-micron particle of any one of claims 1 to 18 or 23 and a pharmaceutically acceptable vehicle.

25. A method of preparing the pharmaceutical composition according to claim 24, the method comprising contacting the sub-micron particle of any one of claims 1 to 18 or 23 with a pharmaceutically acceptable vehicle.

26. The sub-micron particle of any one of claims 1 to 18 or 23, or the pharmaceutical composition of claim 24, for use as a medicament.

27. A vaccine composition comprising the sub-micron particle of any one of claims 1 to 18 or 23, or the pharmaceutical composition of claim 24.

28. The sub-micron particle of any one of claims 1 to 18 or 23, the pharmaceutical composition of claim 24 or the vaccine of claim 27, for use in stimulating an immune response in a subject.
